(19) United States
(12) Patent Application Publication
(10) Pub. No.: US 2025/0376442 A1
(43) Pub. Date: Dec. 11, 2025

(54) HIGH-YIELD PRODUCTION OF EPSILON-CAPROLACTAM FROM NYLON 6 PLASTIC WASTE VIA AMMONIA-ASSISTED DEPOLYMERIZATION

(71) Applicants: Massachusetts Institute of Technology, Cambridge, MA (US); Wisconsin Alumni Research Foundation, Madison, WI (US); Alliance for Sustainable Energy, LLC, Golden, CO (US)

(72) Inventors: Yuriy Roman, Cambridge, MA (US); Gregg Beckham, Golden, CO (US); Shannon Stahl, Madison, WI (US); Xianyuan Wu, Cambridge, MA (US)

(21) Appl. No.: 19/230,822

(22) Filed: Jun. 6, 2025

Related U.S. Application Data
(60) Provisional application No. 63/657,335, filed on Jun. 7, 2024.

(51) Int. Cl. C07D 201/12; C08J 11/26

(52) U.S. Cl. CPC C07D 201/12; C08J 11/26; C08J 2377/02

(57) ABSTRACT

Disclosed are methods for producing ε-caprolactam from nylon 6 plastic waste.

### RELATED APPLICATIONS

This application claims the benefit of priority to U.S. Provisional Patent Application No. 63/657,335, filed Jun. 7, 2024.

### STATEMENT OF GOVERNMENT SUPPORT

This invention was made with government support under DE-AC36-08GO28308 awarded by the U.S. Department of Energy. The government has certain rights in the invention.

### BACKGROUND

Owing to their low cost, versatility, tunability, durability, and lightweight nature, plastics have experienced remarkable market proliferation while significantly improving the quality of human life for over a century. This growth has resulted in an estimated plastic production of 380 million tons annually. Nylon 6, also known as polyamide 6, is a semi-crystalline polymer that is commercially synthesized via the water-assisted or anionic ring-opening polymerization (ROP) of ε-caprolactam using an acylated caprolactam initiator. Due to its excellent strength, durability, and elastic properties, nylon 6 has found a wide range of applications in the textile, automotive, electronics, packaging, fishing, and medical industries. As a result, it is projected to have an annual production rate of more than 10.4 million tons by the year 2027. Unfortunately, due to their exceptional mechanical properties and chemical resistance, coupled with a lack of effective recycling methodologies, nylon 6 materials typically end up in landfills or the ocean (˜10% of total ocean plastics consisting of fishing nets), contributing to the near-catastrophic amount of plastic pollution seen today.

As such, there is a tremendous impetus for the development of efficient and sustainable methodologies for the recycling of nylon 6 plastics to enable a circular economy. Recent years have seen ongoing efforts toward the recycling of nylon 6, predominately focused on mechanical recycling, where the nylon is subjected to melting and reshaping to form usable materials. Despite the mechanical recycling of nylon being both cost-effective and straightforward, it comes with a significant drawback: the partial degradation of the main polymer chain. This results in the deterioration of both the physical and thermal properties of the recycled material compared to pristine nylon. Accordingly, there is an ongoing, unmet need for new methods for producing ε-caprolactam from nylon 6 plastic waste.

### SUMMARY OF THE INVENTION

The disclosed technology includes a route for the efficient depolymerization of nylon 6 which enables high yield production of ε-caprolactam. In certain embodiments, the process functions via a one-pot sequential reaction comprising of ammonolysis and cyclodeamination or alcoholysis. This innovative protocol delivers high yields of ε-caprolactam from industrial nylon 6 using propanol as a solvent and phosphoric acid as a catalyst. The strikingly efficient protocol also demonstrates adaptability to depolymerize nylon 6 from various sources including dyed fishing net, thread, 3-D printing filament, carpet, and t-shirt fabric while achieving a maximum ε-caprolactam yield of 86%. Finally, the upscaled production and subsequent separation of ε-caprolactam is shown, suggesting the process to be industrially compatible.

In one aspect, disclosed herein are methods for producing ε-caprolactam from nylon 6 comprising contacting the nylon 6 with an alcohol and an acid.

In one aspect, disclosed herein are methods for producing ε-caprolactam from nylon 6 comprising contacting the nylon 6 with ammonia and an acid.

### BRIEF DESCRIPTION OF THE DRAWINGS

FIGS. 1A and 1B show an overview of the production of ε-caprolactam from nylon 6.

FIG. 1A shows recent progress based on the literature for the chemical recycling of nylon 6 to ε-caprolactam.

FIG. 1B illustrates the ammonia-assisted depolymerization of nylon 6 to ε-caprolactam via a one-pot sequential reaction consisting of ammonolysis followed by cyclodeamination disclosed herein.

FIGS. 2A-2E show the influence of a range of parameters on the production of ε-caprolactam from nylon 6. Unless otherwise specified, standard reaction conditions were used: 200 mg nylon 6, 0.2 mmol H₃PO₄, 180° C., 5 mL EG, 80 psi NH₃, 18 h.

FIG. 2A is a scheme showing the transformation of Nylon 6 to ε-caprolactam

FIG. 2B shows catalyst screening results (20 mg loading for heterogeneous catalysts, 0.4 mmol NH₄⁺ for ammonium salt and 0.2 mmol for homogeneous Lewis acid catalysts);

b: 200 mg HZSM-5 (1030 μmol acid sites/g) contained a total of 0.206 mmol acid sites c: 600 mg HZSM-5.

FIG. 2C shows a reaction temperature investigation.

FIG. 2D shows reaction time and pressure investigations. Yields were quantified by GC-FID based on calibration curves using a 1,3,5-tri-tert-butylbenzene external standard.

FIG. 2E shows the effects of pressure on product yield.

FIGS. 3A-3C show the elucidation of reaction pathway by means of kinetic analysis.

FIG. 3A shows product concentrations [experimental and modeled] as a function of reaction time. Reaction conditions: 200 mg nylon 6, 0.2 mmol H₃PO₄, 180° C., 5 mL EG, 80 psi NH₃, 0-18 h.

FIG. 3B shows the concentration of 6-aminohexanamide as a function of reaction time during ring closure. Reaction conditions: 0.293 mmol 6-aminohexanamide, 0.033 mmol H₃PO₄, 180° C., 5 mL EG, 80 psi NH₃, 0-60 minutes.

FIG. 3C shows the concentration of ε-caprolactam as a function of time during ring opening. Reaction time: 1.77 mmol ε-caprolactam, 0.2 mmol H₃PO₄, 180° C., 5 mL EG, 80 psi NH₃, 1-2 h. Yields were quantified by GC-FID based on calibration curves using a 1,3,5-tri-tert-butylbenzene external standard.

FIG. 4A shows a plausible reaction mechanism for the acid-catalyzed depolymerization of nylon 6 to produce E-caprolactam involving ammonolysis and subsequent cyclodeamination. Stage 1: Ammonolysis; Stage 2: Cyclodeamination.

FIG. 4B shows the concentration of 6-aminohexanamide as a function of reaction time during ring closure. Reaction conditions: 0.293 mmol 6-aminohexanamide, 0.033 mmol H₃PO₄, 180° C., 5 ml EG, 80 psi NH₃, 0-60 minutes.

FIG. 4C shows the concentration of ε-caprolactam as a function of time during ring opening. Reaction time: 1.77 mmol ε-caprolactam, 0.2 mmol H₃PO₄, 180° C., 5 mL EG, 80 psi NH₃, 1-2 h.

FIG. 4D shows product concentrations (experimental and modeled) as a function of reaction time. Reaction conditions: 200 mg nylon 6, 0.2 mmol H₃PO₄, 180° C., 5 mL EG, 80 psi NH₃, 0-24 h.

FIG. 4E shows a proposed reaction mechanism for the acid-catalyzed ammonolysis of nylon 6 to ε-caprolactam.

FIG. 5 shows an illustrated reaction scheme used for the kinetic modeling of nylon 6 depolymerization as seen in FIG. 3A.

FIG. 6A shows the influence of a range of solvents on the production of ε-caprolactam from nylon 6. Solvent screen results for nylon depolymerization. Unless otherwise specified, standard reaction conditions were utilized: 200 mg nylon 6 powder, 0.2 mmol H₃PO₄, 180° C., 5 mL solvent, 80 psi NH₃, 18 h. Yields were quantified by GC-FID based on calibration curves using a 1,3,5-tri-tert-butylbenzene external standard. ^{a}24 h.

FIG. 6B shows nylon 6 loading screening results for nylon 6 depolymerization, 24 h.

FIG. 6C shows FTIR spectroscopy of crude depolymerized mixture of nylon 6.

FIG. 7 shows the established scope for the depolymerization of real-world nylon 6 feedstocks to ε-caprolactam. General reaction conditions: 200 mg nylon 6, 0.2 mmol H₃PO₄, 180° C., 5 mL propanol, 80 psi NH₃, 24 h. The nylon 6 filament contained 75% of nylon 6, with the remaining material being glass fiber. The t-shirt fabric contained 97% nylon 6. The plastic mixture contained a mixture of fishing net (both green and white, PP, and low-density PE). Yields were quantified by GC-FID based on calibration curves using a 1,3,5-tri-tert-butylbenzene external standard.

FIGS. 8A-8C show the upscaling the production and separation of ε-caprolactam from fishing net.

FIG. 8A shows general reaction conditions: 180° C., 80 psi NH₃, 24 h, i) for 200/400 mg fishing net (white): 0.2 mmol H₃PO₄, 6 mL propanol; ii) for 500/600 mg fishing net (white): 0.4 mmol H₃PO₄, 6 mL propanol; iii) for 800 mg fishing net (white): 0.6 mmol H₃PO₄, 7 mL propanol.

FIG. 8B shows 1. Parallel reaction using 600 mg fishing net; 2. Separation of ε-caprolactam by a short path silica gel filtration using a solvent mixture [1 MeOH:10 EtOAc].

FIG. 8C shows NMR spectra of the product obtained from the upscaling process.

FIG. 9 shows the reaction pressure test as a function of time. Reaction conditions: 600 mg fishing net (Green), 0.4 mmol H₃PO₄, 80 psi NH₃, 180° C., 24 h, 6 mL propanol.

FIG. 10 GC-FID spectrum of crude depolymerized mixture. Reaction conditions: 200 mg nylon 6 powder, 0.2 mmol H₃PO₄, 80 psi NH₃, 180° C., 18 h, 5 mL EG.

FIG. 11 shows GC-FID spectrum of crude depolymerized mixture. Reaction conditions: 600 mg fishing net (white), 0.4 mmol H₃PO₄, 80 psi NH₃, 180° C., 24 h, 6 mL propanol.

FIGS. 12A-12G show the influence of reaction conditions on nylon 6 depolymerization. Unless otherwise specified, the following reaction conditions were used: 200 mg nylon 6 pellet (Sigma-Aldrich), 0.2 mmol H₃PO₄, 220° C., 5 mL n-PrOH, 2 h.

FIG. 12A shows the reaction scheme and associated products.

FIG. 12B shows catalyst screening results, HCl: 0.6 mmol, H₂SO₄: 0.3 mmol, others: 0.2 mmol.

FIG. 12C shows solvent screening results.

FIG. 12D shows temperature screening results.

FIG. 12E shows reaction time effects (200° C.).

FIG. 12F shows nylon 6 loading screening results (210° C., 2.5 h).

FIG. 12G shows catalyst loading with respect to nylon 6 screening results (210° C., 2.5 h). Yields were quantified by gas chromatography with flame ionization detection (GC-FID) based on calibration curves using a 1,3,5-tri-tert-butylbenzene external standard.

FIGS. 13A-13E shows the elucidation of reaction pathway by means of model experiments and kinetic analysis.

FIG. 13A shows the initial reaction scheme used for the kinetic modeling of nylon 6 depolymerization.

FIG. 13B Concentration of propyl 6-aminohexanoate as a function of reaction time during ring closure. Reaction conditions: 0.293 mmol propyl 6-aminohexanoate, 0.033 mmol H₃PO₄, 180° C., 5 ml n-PrOH, 0-0.5 h.

FIG. 13C shows the concentration of ε-caprolactam as a function of time during ring opening. Reaction conditions: 1.77 mmol ε-caprolactam, 0.2 mmol H₃PO₄, 200° C., 5 mL n-PrOH, 0.5-1 h.

FIG. 13D shows product concentrations [experimental and modeled] as a function of reaction time. Reaction conditions: 200 mg nylon 6, 0.2 mmol H₃PO₄, 200° C., 5 mL n-PrOH, 0-6 h.

FIG. 13E shows the proposed reaction mechanism for nylon 6 depolymerization via acid-catalyzed alcoholysis.

FIG. 14 shows a simplified process flow diagram with identified process improvements for depolymerization of nylon 6 to ε-caprolactam via alcoholysis.

FIGS. 15A-15G shows the total installed capital (TCI), OPEX, MSP breakdown, GHG impact, and sensitivity analysis.

FIG. 15A shows installed capital breakdown by process area. The outside battery limit is abbreviated as OSBL.

FIG. 15B shows OPEX breakdown by process area.

FIG. 15C shows MSP breakdown for overall nylon 6 depolymerization process.

FIG. 15D shows univariate sensitivity analysis results for the minimum selling price of r-nylon 6. The shaded area in grey represents the uncertainty range in the MSP, estimated by Monte-Carlo simulation for 1,000 iterations. The 5-year average market price of v-nylon 6 is shown for reference.

FIG. 15E shows univariate sensitivity analysis results for GHG emissions for r-nylon 6 across various process parameters. The emissions for v-nylon 6 is listed for reference.

FIG. 15F shows cradle-to-gate LCA results for selected impact categories for the base case, and virgin nylon 6 production.

FIG. 15G shows a comparison of GHG emissions of nylon 6 depolymerization via alcoholysis for decarbonization scenarios including the use of renewable electricity (ReEDS Mid-case scenario with 95% decarbonization by 2035) and renewable heat (from renewable natural gas).

FIG. 15H shows the GHG potential impact for the proposed design broken down by component contribution. Virgin production of nylon 6 is listed for reference.

FIG. 16 shows the FTIR spectroscopy of crude depolymerized mixtures derived from nylon 6. Reaction conditions: 200-600 mg nylon 6 pellet, 0.2 mmol H₃PO₄, 210° C., 2.5 h, 5 mL n-PrOH. The —C═O and —N—H stretching vibrations of amide group at 1636 and 1535 cm⁻¹ were previously reported in literature.

FIG. 17 shows the ¹H NMR of a crude depolymerized mixture derived from nylon 6. Reaction conditions: 200-600 mg nylon 6 powder, 0.2 mmol H₃PO₄, 210° C., 2.5 h, 5 mL propanol.

### DETAILED DESCRIPTION OF THE INVENTION

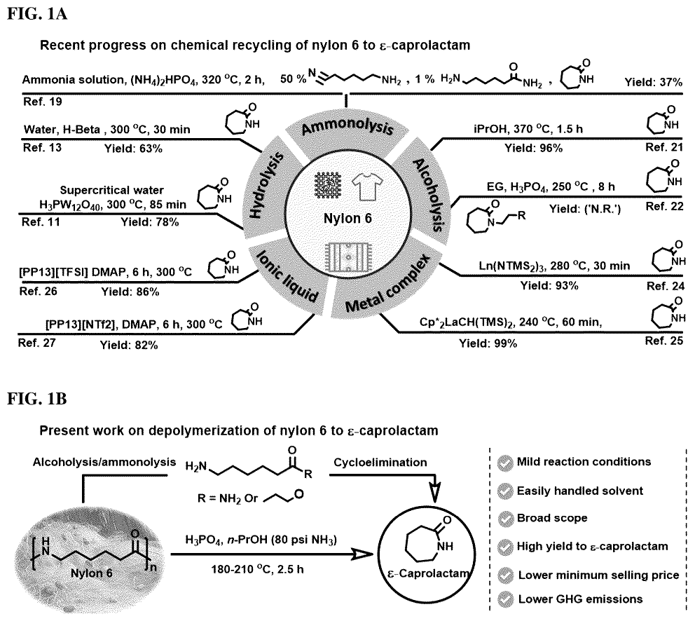

To circumvent drawbacks associated with the mechanical recycling of nylon 6, recent efforts have been devoted to the development of highly efficient chemical recycling methods utilizing hydrolysis, pyrolysis, ammonolysis, alcoholysis, hydrogenolysis, organic metal complexes, and ionic liquid-assisted depolymerization. These methods are capable of selectively depolymerizing nylon 6 into its original precursor, ε-caprolactam, which can be reused to produce pristine nylon 6, thereby enabling closed-loop recyclability. However, these processes all require high reaction temperatures and pressures [FIG. 1A]. For example, a) the hydrolysis of nylon 6 in supercritical water over a strong acid H₃PW₁₂O₄₀ catalyst led to 78% yield of ε-caprolactam while requiring a temperature of 300° C.; b) the ammonolysis of nylon 6 delivered a low yield of 37% to ε-caprolactam using an (NH₄)₂HPO₄ catalyst and ammonia/water at 320° C.; c) the alcoholysis of nylon 6 gave 93% yield of ε-caprolactam in in supercritical isopropanol at 370° C. for 90 minutes; d) the treatment of nylon 6 in ionic liquids containing 4-dimethylaminopyridine (DMAP) at 300° C. yielded between 55% and 85% of ε-caprolactam; and e) the organic metal complex Ln^{NTMS} mediated depolymerizations of nylon 6 and generated an excellent yield of 93% to E-caprolactam at 280° C.

Alternative to prior works carried out using harsh reaction conditions, in this work, we describe an elegant, powerful catalytic protocol which involves a one-pot sequential reaction consisting of ammonolysis followed by cyclodeamination. This allows for the mild and highly selective ammonia-assisted depolymerization of nylon 6 into ε-caprolactam at excellent yields while using environmentally benign propanol as solvent and H₃PO₄ as a catalyst at a low reaction temperature of 180° C. The significance of this method lies in its high efficiency, scalability, and broad applicability to various sources of ‘real world’ nylon 6 plastic wastes, including fishing nets, thread, 3-D printing filament, fabric, and carpet, all of which are shown to be efficiently converted to ε-caprolactam at impressive yields.

In one aspect, disclosed herein are methods for producing ε-caprolactam from nylon 6, comprising contacting the nylon 6 with an alcohol and an acid.

In certain embodiments, the alcohol is methanol, ethanol, n-propanol, isopropanol, n-butanol, or ethylene glycol. In certain embodiments, the alcohol is propanol.

In one aspect, disclosed herein are methods for producing ε-caprolactam from nylon 6, comprising contacting the nylon 6 with ammonia and an acid.

In certain embodiments, the ammonia is at a pressure of more than 60 psi. In certain embodiments, the ammonia is at a pressure of about 60 psi, about 80 psi, about 100 psi, or about 120 psi. In certain embodiments, the ammonia is at a pressure of about 80 psi.

In certain embodiments, the nylon 6 is nylon 6 plastic waste. In certain embodiments, the plastic waste is nylon 6 powder, nylon 6 film, nylon 6 pellets, green nylon 6 fishing net, white nylon 6 fishing net, nylon 6 thread, nylon 6 clothing, nylon 6 carpet, or nylon 6 filaments.

In certain embodiments, the acid is a Lewis acid. In other embodiments, the acid is a Brønsted acid. In certain embodiments, the acid is a mineral acid. In certain embodiments, the acid is HCl, H₂SO₄, H₃PO₄, (NH₄)₂HPO₄, Sn(OTf)₂, Ce(OTf)₄, or La(OTf)₃. In certain embodiments, the acid is phosphoric acid (H₃PO₄).

In certain embodiments, the acid is present about from 1 wt % to about 25 wt %. In certain embodiments, the acid is present at about 5 wt %, about 10 wt %, about 15 wt %, about 20 wt %, or about 25 wt %.

In certain embodiments, the method is performed in a protic solvent. In certain embodiments, the method is performed in an alcoholic solvent. In certain embodiments, the method is performed in methanol, ethanol, n-propanol, isopropanol, n-butanol, or ethylene glycol. In certain embodiments, the method is performed in propanol.

In certain embodiments, the method is performed at about 150° C., about 160° C., about 170° C., about 180° C., about 190° C., about 200° C., about 210° C., about 220° C., about 230° C., or about 240° C. In certain embodiments, the method is performed at about 180° C.

In certain embodiments, the method is performed for more than about 4 hours, more than about 5 hours, or more than about 6 hours. In certain embodiments, the method is performed for about 4 hours, about 5 hours, about 6 hours, or about 7 hours.

In certain embodiments, the method produces ε-caprolactam at a yield of great than about 70%. In certain embodiments, the method produces ε-caprolactam at a yield of about 70%, about 75%, about 80%, about 85%, or about 90%.

### Definitions

Unless otherwise defined herein, scientific and technical terms used in this application shall have the meanings that are commonly understood by those of ordinary skill in the art.

The methods and techniques of the present disclosure are generally performed, unless otherwise indicated, according to conventional methods well known in the art and as described in various general and more specific references that are cited and discussed throughout this specification.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amido”, as used herein, refers to a group

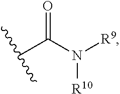

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

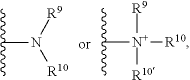

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

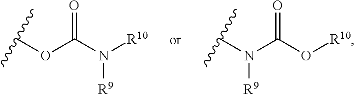

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamido” is art-recognized and refers to the group represented by the general formulae

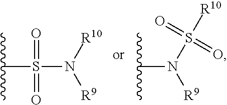

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

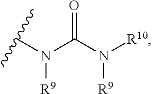

wherein R⁹ and R¹⁰ independently represent hydrogen or a hydrocarbyl.

### EXAMPLES

The invention now being generally described, it will be more readily understood by reference to the following examples which are included merely for purposes of illustration of certain aspects and embodiments of the present invention and are not intended to limit the invention.

### Example 1: Synthesis of Exemplary Polymers

### Chemicals and Solvents

ε-Caprolactam (99%) was purchased from Sigma-Aldrich. 6-aminohexanamide (95%) was purchased from Hangzhou Molcore BioPharn. Methanol (99.9%) was purchased from VWR chemicals. Anhydrous ethanol was purchased from Koptec. Hexane (AR/ACS) was purchased from Macron Fine Chemicals. Propanol (99.9%), isopropanol (99.9%), ethylene glycol (99%), glycerol (99%), THF (99.9%), acetone (99.9%) and ethyl acetate (99.9%) were purchased from Sigma-Aldrich. Nylon 6 powder and film were obtained from Goodfellow. Nylon pellets were purchased from Sigma-Aldrich. Fishing net, thread, T-shirt, carpet, and filament were purchased from Amazon.

### Catalysts

Anhydrous NH₃ (99.99%) was supplied by Airgas. H₃PO₄ (85%) aqueous solution, (NH₄)₂HPO₄ (98%) and NH₄Cl (99.5%) were purchased from Sigma-Aldrich. Lanthanum(III) trifluoromethanesulfonate (99%), Tin(II) trifluoromethanesulfonate (98%), and Cerium(IV) trifluoromethanesulfonate (98%) were purchased from Fisher Scientific. HZSM-5 was purchased from Zeolyst and calcined at 550° C. before use.

### General Experimental Procedures

Ammonia-assisted depolymerization of Nylon 6 materials in presence of a H₃PO₄ catalyst was carried out in 25 mL high-pressure Parr autoclave, equipped with a Teflon magnetic stirring bar. Typically, the autoclave was charged with 600 mg Nylon 6 substrate (fishing net), 0.4 mmol H₃PO₄, 6 mL propanol. The reactor was sealed and pressurized with 80 psi NH₃ at room temperature. The reactor was heated to 180° C. and stirred at 500 rpm for 24 h. After completion of the reaction, the reactor was quenched in an ice bath. Then 40 ml methanol and approximately 30 mg of 1,3,5-tri-tert-butyl benzene as external standard were added to crude depolymerized mixture. The suspension was subjected to centrifugation to separate liquids and methanol insoluble fractions. The liquid products were identified using GC coupled to a mass spectrometer and then quantified off-line using a GC-FID. The mixture rich in ε-caprolactam was eventually isolated by a short path silica gel filtration using a mixture of solvents (1:10 vol. MeOH:EtOAc).

### ε-Caprolactam Quantification

ε-Caprolactam yield was by GC-FID based on calibration curves using a 1,3,5-tri-tert-butylbenzene external standard, using the following formula:

$$ε - Caprolactam yield ( \% ) = \frac{Molar of ε - caprolactam obtained experimentally}{Theorectial molar of ε - caprolactam obtained from nylon 6} \times 100 \%$$

### Results and Discussion

### Establishing Optimized Reaction Conditions for Depolymerizing Nylon 6

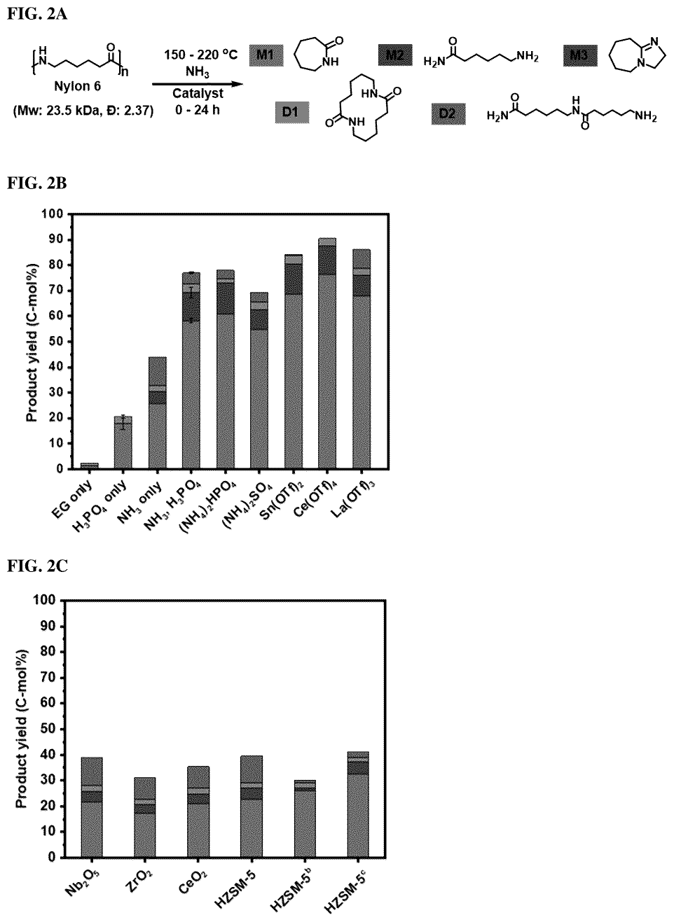

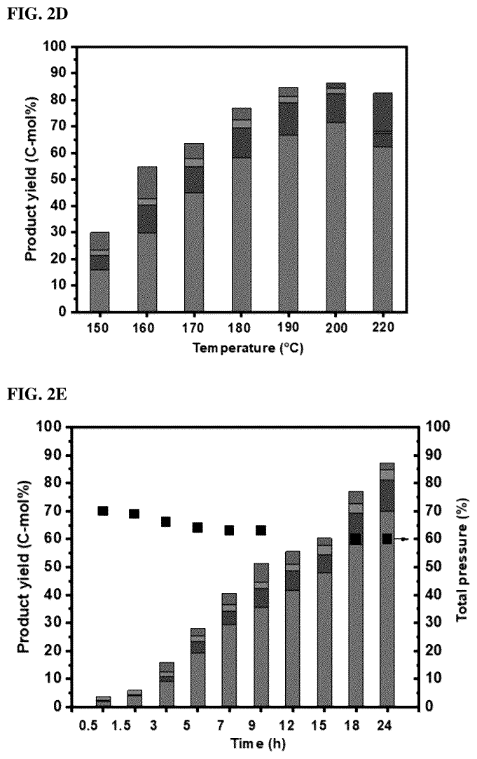

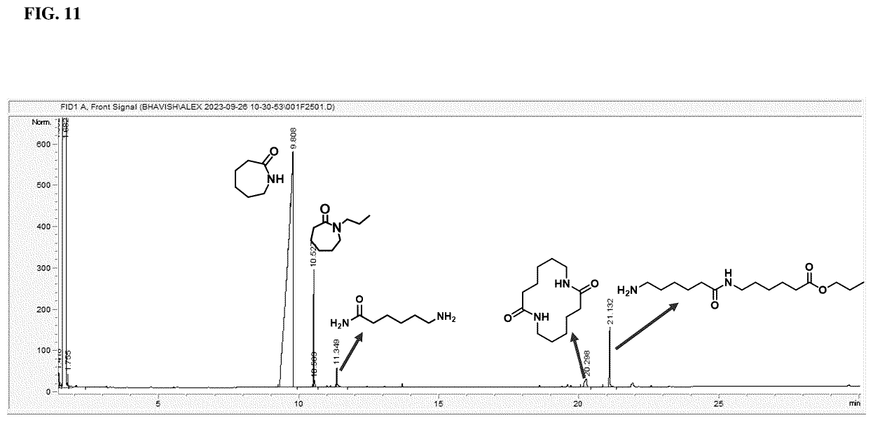

We initially hypothesized that an acid catalyst could serve as an active center for both ammonolysis and cyclodeamination by coordinating with carbonyl oxygens, thereby making them more susceptible to nucleophilic attack by ammonia or amine intermediates. To this end, a range of different acid catalysts, including both heterogeneous and homogeneous Lewis and Brønsted acids, were initially tested with ethylene glycol (EG) as a solvent for the one-pot ammonia-assisted depolymerization of nylon 6 to ε-caprolactam at a low reaction temperature of 180° C. [FIG. 2A]. EG was selected due to its ability to fully dissolve nylon 6 while maintaining a homogeneous reaction medium at 180° C., thereby facilitating the efficient depolymerization of nylon 6. Pure glycolysis of nylon 6 was ineffective, which is consistent with the previously reported results. However, in the presence of H₃PO₄, the yield of ε-caprolactam was improved to 18%, implying the presence of an acid catalyzes glycolysis while also potentially aiding in subsequent cyclodehydration. Meanwhile, using ammonia gas alone contributed to a slightly higher yield of 25.7% to ε-caprolactam, demonstrating efficient nucleophilic attack. The use of ammonia also produces a 6-aminohexanamide intermediate which is favorable for cyclodeamination, which can lead to higher overall yields of ε-caprolactam relative to glycolysis alone. The cyclic dimer, namely 1,8-diazacyclotetradecane generated from the deamination of linear dimer was also observed in small quantities (For product identification sees FIG. 10), which can also likely be subsequently converted by amination to ε-caprolactam via nucleophilic attack with NH₃.

Encouragingly, the combination of ammonia and the H₃PO₄ catalyst dramatically promoted the overall yield of ε-caprolactam to 58.2%, implying a synergistic effect in which the propensity of the ammonia toward nucleophilic attack of the electrophilic carbonyl carbon is assisted by the presence of an acid catalyst. However, the neutralization of H₃PO₄ (0.6 mmol H⁺ assuming complete dissociation) by excess ammonia (80 psi, an equivalent amount of at ˜5.81 mmol assuming an ideal gas) likely occurs immediately, leading to the formation of NH₄⁺ ions. Nevertheless, neutralization doesn't necessarily result in the immediate loss of catalytic effects. On the contrary, NH₄⁺ ions are still capable of coordinating with carbonyl groups, thereby promoting subsequent nucleophilic attack by ammonia to form a mechanistic sequence leading to enhanced depolymerization efficiency. This was confirmed by instead using a phosphate salt [(NH₄)₂HPO₄] and NH₄Cl, both of which led to a similar ε-caprolactam yield as the ammonia-acid system. Interestingly, homogeneous Lewis acid catalysts [e.g. Sn(OTf)₃, Ce(OTf)₄, and La(OTf)₃] also led to high yields of ε-caprolactam, ranging from 60-75%, implying a strong interaction between homogeneous Lewis acid centers and the amide groups in nylon 6. These results are consistent with previous work where M(OTf)ₓ was demonstrated to be active for the depolymerization of polyamide 6,6 [32]. Meanwhile, heterogeneous Brønsted and Lewis acid catalysts, such as ZrO₂, CeO₂, Nb₂O₅ and HZSM-5, all showed poor catalytic reactivity, leading to ε-caprolactam yields of only ˜20% [FIG. 2B]. Further increasing the loading of HZSM-5 (600 mg) does not result in obvious increases to the yield of ε-caprolactam. This highlights the challenges associated with heterogeneous acid catalysts for the depolymerization of nylon 6, as despite their high loading and inherent acidity, poor depolymerization efficiency resulted. The observed lack of reactivity could be attributed to either unfavorable surface interactions with the amide bonds in nylon 6 or diffusion limitations.

Considering the low relative cost and required mass ratio (1:10) of catalyst to substrate, we elected to further investigate the influence of reaction temperature on nylon 6 depolymerization using H₃PO₄ [FIG. 2C]. The reaction performed at 150° C. delivered a yield of ˜16% toward ε-caprolactam. Meanwhile, temperatures beyond 200° C. led to yields of approximately 71%. This promoted effect is likely due to the higher temperature allowing the system to more easily overcome the requisite activation barrier, thereby leading to faster depolymerization. Next, the influence of reaction time was investigated [FIG. 2D]. The production of ε-caprolactam is significantly favored at longer reaction times, reaching a ε-caprolactam yield of close to 70% at 24 h, coupled with a slight increase in the production of a 6-aminohexanamide intermediate. It is worth noting that overall pressure slowly drops by around 10 psi for both the case using 200 mg and 600 mg as starting substrate (FIG. 2D), indicating ammonia is not largely consumed due to auto-supplementation via the cyclodeamination of 6-aminohexanamide within the reaction cycle. The loss of ammonia probably occurs in the form of linear oligomers and 6-aminohexanamide which have not been fully converted to ε-caprolactam.

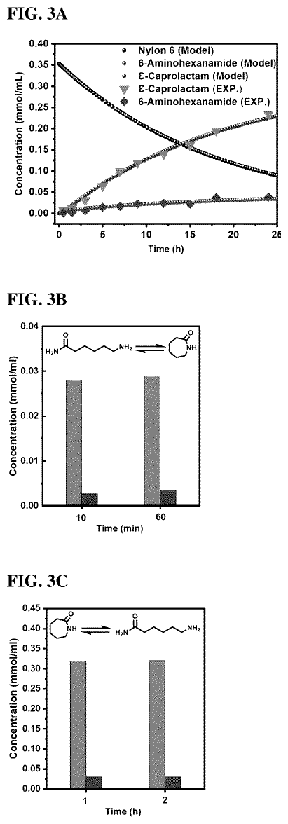

Mechanistic Insights into the Depolymerization of Nylon 6
To shed light on the reaction mechanism and kinetics for the depolymerization of nylon 6, a series of probe reactions on individual compounds was completed using the reaction conditions previously described [FIG. 3]. It was found that the nylon 6 to ε-caprolactam reaction likely occurs predominantly via a 6-aminohexanamide intermediate [FIG. 2D, 3A], as pure 6-aminohexanamide is capable of undergoing fast cyclodeamination to ε-caprolactam within a 10-minute reaction period, with an impressive conversion of 95% [FIG. 3B]. Further increasing the reaction time led to no significant variation in the reagent and product concentrations, implying an equilibrium between 6-aminohexanamide and ε-caprolactam. This was further verified by using pure ε-caprolactam as a probe, which was converted back into 6-aminohexanamide at low yields under identical reaction conditions [FIG. 3C].

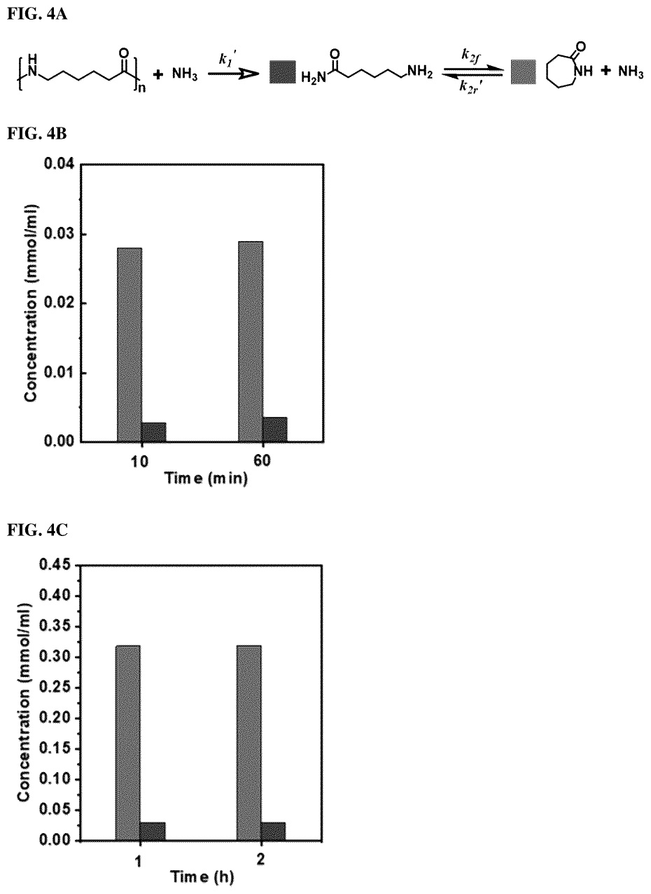

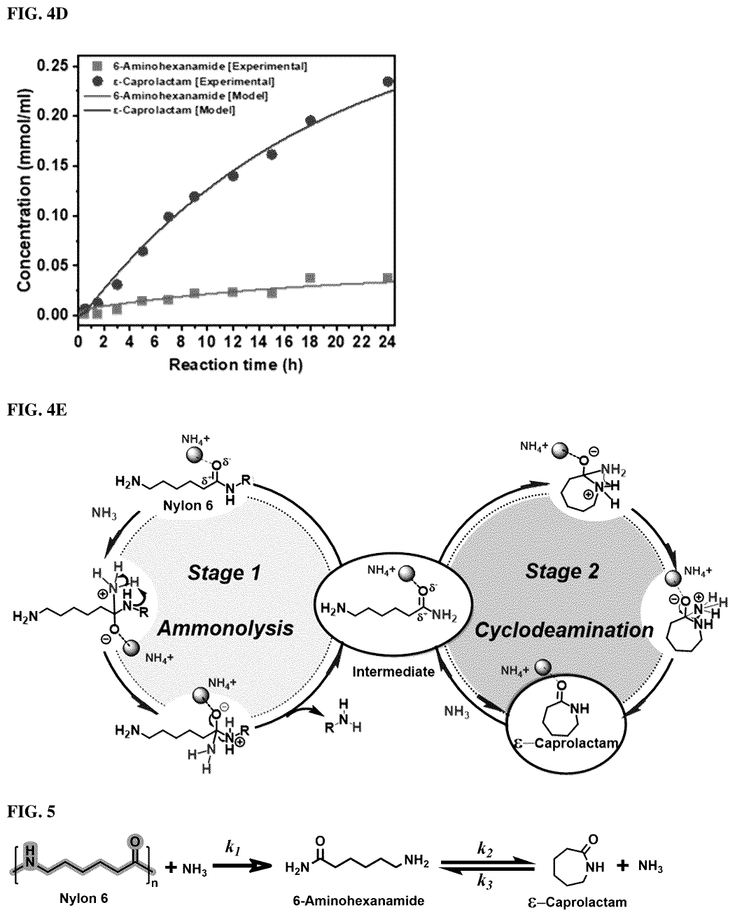

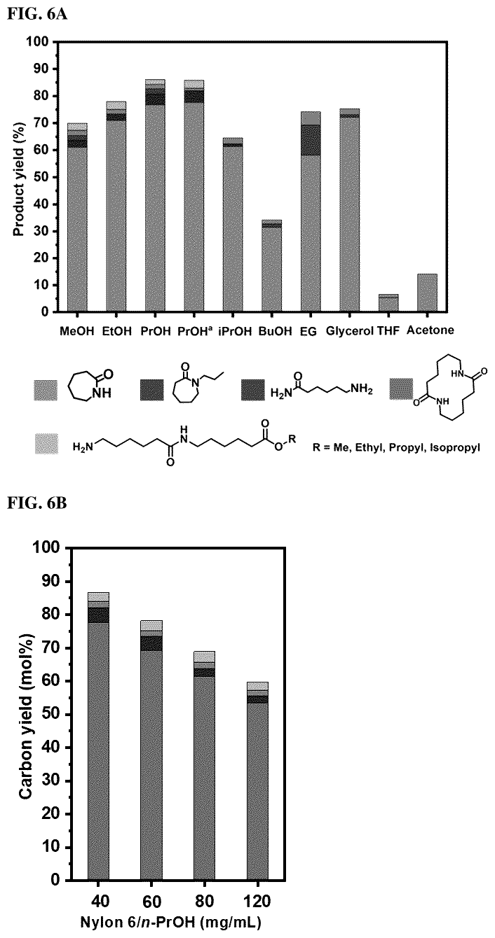

Based on the product distributions, a plausible acid-catalyzed reaction mechanism for the ammonia-assisted depolymerization of nylon 6 involving two consecutive steps of ammonolysis and cyclodeamination is proposed, as described in FIG. 4. In Stage 1, the amide bond of nylon 6 is initially coordinated and activated by the Brønsted acidic NH₄⁺, making it more susceptible to nucleophilic attack by ammonia gas to produce 6-aminohexanamide, which then immediately undergoes cyclodeamination during Stage 2 to yield the final product of ε-caprolactam, with ammonia gas being regenerated in the reaction cycle.

To model the kinetics of the depolymerization reaction, we used the system found in Scheme 1 and made a number of simplifying assumptions: a) the depolymerization of nylon 6 proceeds via a pseudo-homogeneous, first-order kinetic model, b) the NH₃ concentration in liquid phase is approximately constant, and as a result the effects of ammonia are lumped into the apparent kinetic rate constants (kₙ′=kₙC(ammonia)), and c) the equilibrium between nylon 6 depolymerization and repolymerization is negligible. Based on these assumptions, the system can be approximated using a power-law model,

By fitting the experimental concentrations of ε-caprolactam and 6-aminohexanamide as a function of time to a first-order power law model [FIG. 3A], the apparent rate constants for the reaction system were calculated. The constants, k₁′, k₂, and k₃′ were thus determined as 0.0536, 2.6407, and 0.3763 h⁻¹, respectively, further reinforcing experimental observations demonstrating the ammonolysis of nylon 6 to be rate-determining.

### Solvent Optimization to Improve Depolymerization Efficiency

In light of the challenge posed by the separation of ε-caprolactam and ethylene glycol, we next investigated the possibility of using alternative polar protic/aprotic solvents with the potential to offer better compatibility with the catalyst, substrate, and overall process (FIG. 5). Treating nylon 6 with glycerol led to an improved yield of 72% as compared with EG, however, similar (if not worse) separation challenges exist for glycerol as for ethylene glycol. As for volatile alcohols, methanol led to a similar yield as ethylene glycol (61.1%). Meanwhile, larger protic alcohols led to increased yields: ethanol generated a yield of 71.0% while n-propanol generated an impressive yield of 76.8% (For product identification sees 2, FIG. 11). This is in line with the observations where under supercritical reaction conditions, the yield of ε-caprolactam obtained from the depolymerization of nylon 6 increased with the length of the alkyl chain for primary alcohols. Using isopropanol did not contribute to the yield further, resulting in a yield of approximately 61.4%. These observed differences in reactivity could be related to differences in solvent-substrate interactions (e.g., hydrogen bonding donating or accepting propensity). Such interactions could potentially enhance the electrophilicity of carbonyl groups or the nucleophilicity of ammonia, thereby facilitating the cleavage of amide bonds in nylon 6. Notably, further extending the reaction time to 24 hours in the propanol solvent does not result in a significant increase in the yield of ε-caprolactam, implying a potential equilibrium within the depolymerization step.

In a marked contrast, n-butanol led to an undesirable yield of approximately 30% to ε-caprolactam, suggesting its increased hydrophobicity may be inhibiting its ability to interact with the amide groups in nylon 6. Further demonstrating the need for strong solvent-substrate interactions, polar aprotic solvents including THF, and acetone exhibited poor nylon depolymerization performance. The nature of these solvents likely doesn't facilitate the necessary solubilization or coordination behavior required for efficient depolymerization, thereby resulting in low yields of ε-caprolactam.

### Depolymerization of ‘Real World’ Nylon Feedstocks to ε-Caprolactam

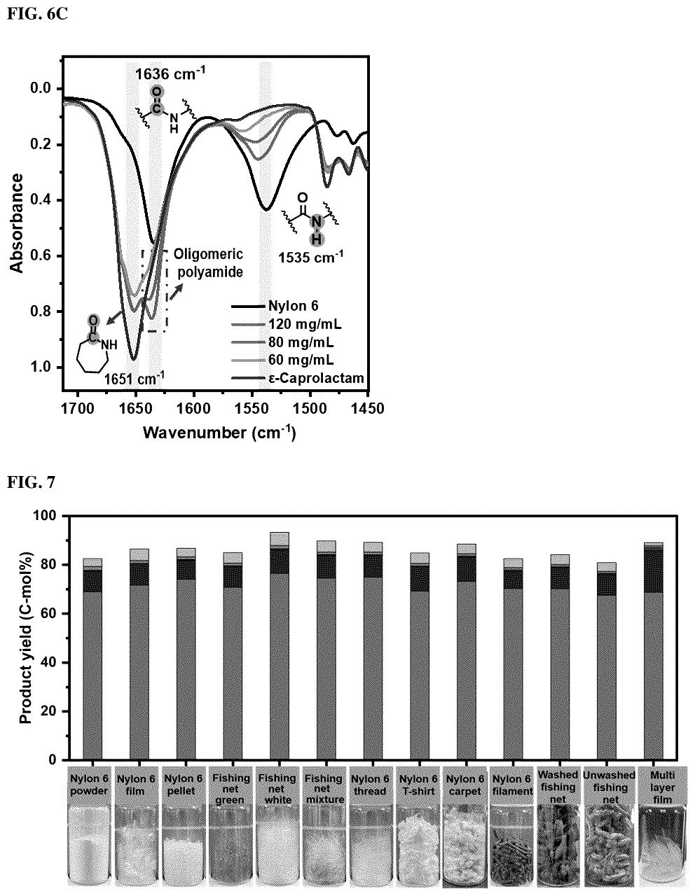

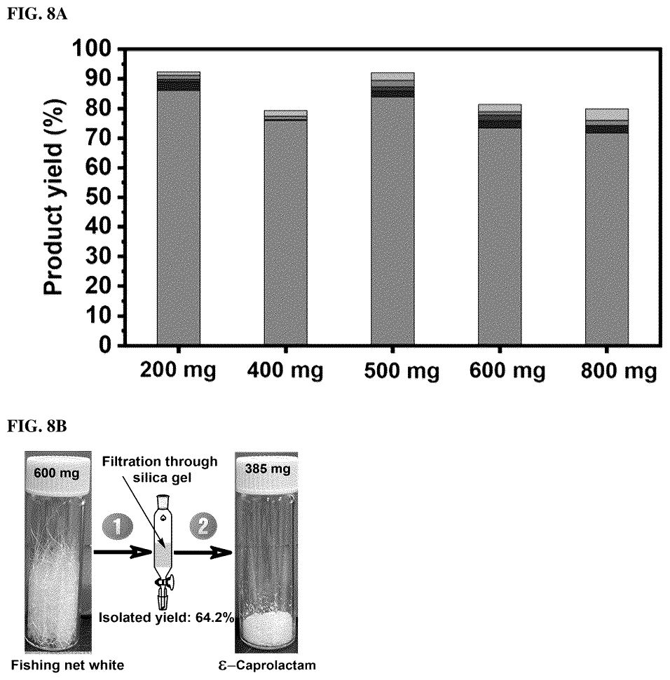

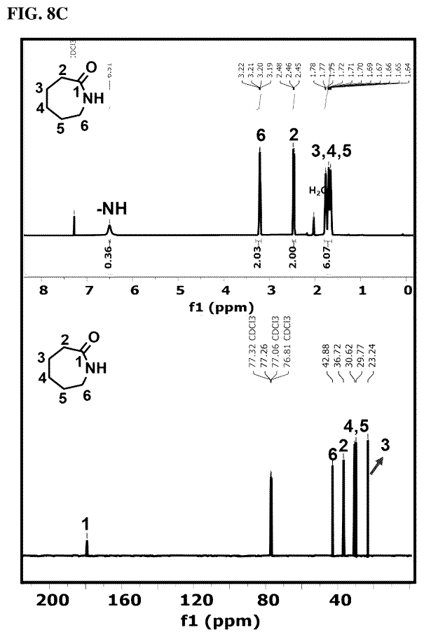

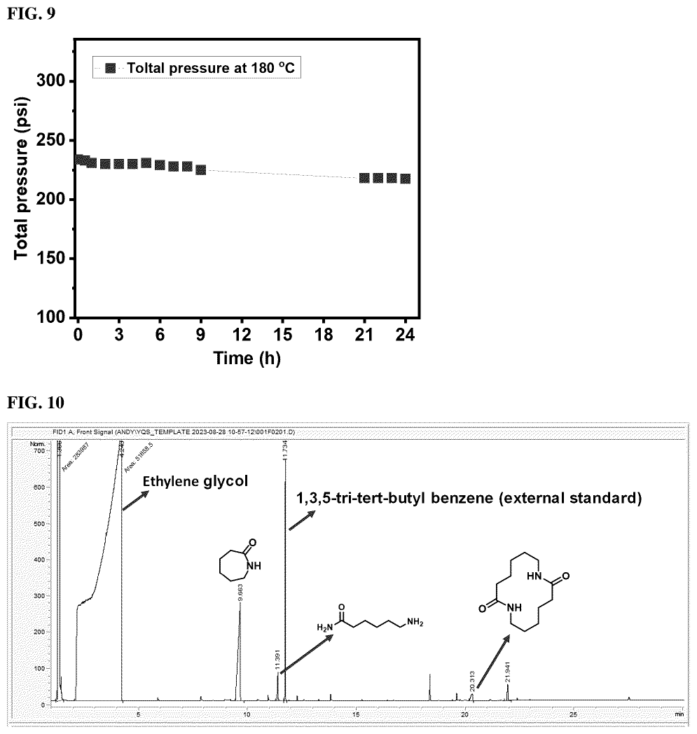

Following optimization, we expanded our catalytic methodology to a set of ‘real world’ nylon 6 plastic wastes including commercial fishing nets (green and white), 3D printing filament, film, t-shirt fabric, carpet, and thread, all of which were chopped into smaller pieces (1 mm length) before being loaded into reactors. Nylon 6 pellets and film both delivered high yields of over 80% to ε-caprolactam under optimized reaction conditions [FIG. 7]. Remarkably, the depolymerization of fishing net (green, white, or a mixture of both), a significant contributor to plastic waste pollution with an estimated annual contribution exceeding 600,000 tons disposed annually into the ocean, achieved the highest yield of up to 86% to ε-caprolactam. Apart from processing common nylon waste, this promising result suggests our process to be resilient in the presence of other components such as dyes found in the majority of common plastic wastes.

We next applied the system to carpet textile, with an estimated annual production of 12 billion feet with approximately 95% ending up in landfills at end-of-life. Using the standard reaction conditions, the carpet material was converted into ε-caprolactam at a yield of 84.8%. Meanwhile, nylon thread, predominately used in the production of textiles, fabrics and clothing items, also resulted in an excellent yield of 85.4%. Finally, nylon t-shirt fabric and 3D printing filament comprising 25 wt. % glassfiber resulted in ε-caprolactam yields of 74.7 and 80.1%, respectively.

In addition to nylon, ocean plastics are predominantly composed of polyolefins, specifically polyethylene (PE) and polypropylene (PP). To assess the suitability of our methodology toward processing mixed plastic waste streams, a mixture of PE, PP, and commercial fishing net material was subjected to our depolymerization process. This resulted in the selective depolymerization of the nylon 6 materials at an impressive yield of 83.6% ε-caprolactam. Meanwhile, the polyolefins were quantitively retained as solids in the post-reaction mixture.

Upscaling the Production and Separation of ε-Caprolactam from Fishing Net

Having established optimal reaction conditions, we proceeded with upscaling production of ε-caprolactam via the selective depolymerization of real-world nylon 6 wastes. To this end, the upscaling of fishing net (400 mg) depolymerization while holding all reaction parameters constant led to slightly decreased yield (75.9%) to ε-caprolactam, lower than that obtainable from 200 mg substrate (86%). Nevertheless, by doubling the H₃PO₄ catalyst loading, a yield of ˜84% toward ε-caprolactam was obtained from 500 mg of nylon fishing net. Further increasing to 600 and 800 mg of fishing net did not significantly compromise the system's depolymerization efficiency, maintaining yields >70%. The crude depolymerized reaction mixture from the 600 mg experiment was purified via short path silica gel filtration using gradient elution (1:10 volume ratio of methanol:EtOAc), generating an isolated yield of 64.2%. It should be noted that this recovery could likely be improved through the use of industrial separation techniques.

### Exemplary Advantages and Improvements Over Existing Methods and Materials

Nylon 6 is a valuable and durable material and recycling it into ε-caprolactam allows for the extraction of a key building block without depleting new resources.

The work presented herein demonstrates a mild, effective, ammonia-assisted strategy for the depolymerization of nylon 6 using a H₃PO₄ catalyst. This allows for the selective production of ε-caprolactam at excellent yields with operating temperatures as low as 180° C. using environmentally benign and easily handled propanol as solvent, in contrast to the traditional method which typically involves operating temperature exceeding 300° C.

The methodology demonstrated here can find adaptability to depolymerize nylon 6 from various sources including dyed fishing net, thread, 3-D printing filament, carpet, and t-shirt fabric while achieving a maximum ε-caprolactam yield of 86%. Moreover, this method allows for the processing of plastic waste mixtures containing both PE and PP as the nylon is selectively depolymerized, thereby leaving behind unreacted polyolefins.

Upscaled production and product separation were successfully demonstrated without significantly compromising yields.

### Exemplary Commercial Applications

Recycling nylon 6 into its building blocks waste reduces the environmental burden associated with traditional disposal methods, such as landfilling or incineration. It helps in minimizing pollution and conserving energy compared to the production of virgin caprolactam.

The conversion of nylon 6 waste to ε-caprolactam contributes to a circular economy by closing the loop on material usage. It promotes sustainability by reusing materials in a closed system rather than following a linear, take-make-dispose model.

Recycling nylon 6 waste into ε-caprolactam can lead to economic advantages by creating a sustainable supply chain, reducing the need for virgin materials, and potentially lowering production costs.

### Characterization Results of Commercial Nylon 6 Materials

TABLE S1
Detailed characterization results for commercial nylon 6 materials

| Nylon 6 | | Mw^{[a]} | Mn^{[a]} | T_{g}^{[b]} | Tₘ^{[b]} | Onset^{[c]} |
|---|---|---|---|---|---|---|
| materials | Compositions | (kDa) | (kDa) | (° C.) | (° C.) | (° C.) |
| Powder | Nylon 6 | 23.5 | 9.9 | 48 | 262 | 417 |
| Film | Nylon 6 | 38.1 | 20.6 | 65 | 226 | 437 |
| Pellet | Nylon 6 | 39.9 | 26.7 | 51 | 224 | 427 |
| Fishing net | Nylon 6 | 32.5 | 21.0 | 53 | 224 | 429 |
| (Green) | | | | | | |
| Fishing net | Nylon 6 | 29.9 | 20.6 | 53 | 223 | 425 |
| (white) | | | | | | |
| Thread | Nylon 6 | 40.0 | 28.2 | 52 | 219.6 | 427 |
| T-shirt | 97% nylon 6 | 44.3 | 26.7 | 53.2 | 225.2 | 421 |
| | 3% spandex | | | | | |
| Carpet | nylon 6 | 31.9 | 22.5 | 51.6 | 223.4 | 421 |
| Filament^{[d]} | 75% nylon 6 | N.D. | N.D. | N.D. | 219 | 422 |
| | 25% glass | | | | | |
| | fiber | | | | | |

| | | | Residue | | Moisture | Nylon 6 |
|---|---|---|---|---|---|---|
| Nylon 6 | | T_{d50%}^{[c]} | Loss^{[c]} | Crystallinity^{[b]} | content | Content |
| materials | Compositions | (° C.) | (%) | (%) | (%) | (%) |
| Powder | Nylon 6 | 447 | 2.6 | 35.8 | 2.5 | 94.9 |
| Film | Nylon 6 | 467 | 0.1 | 22.1 | 1.86 | 98.04 |
| Pellet | Nylon 6 | 459 | 0 | 28.2 | 0.44 | 99.56 |
| Fishing net | Nylon 6 | 457 | 0.1 | 32.9 | 3.35 | 96.55 |
| (Green) | | | | | | |
| Fishing net | Nylon 6 | 455 | 0 | 32.1 | 3.2 | 96.8 |
| (white) | | | | | | |
| Thread | Nylon 6 | 455 | 0 | 32.5 | 2.59 | 97.41 |
| T-shirt | 97% nylon 6 | 455 | 2.7 | N.D. | 1.8 | 95.5 |
| | 3% spandex | | | | | |
| Carpet | nylon 6 | 455 | 1.5 | 28.8 | 3.04 | 95.46 |
| Filament^{[d]} | 75% nylon 6 | 456 | 26.0 | 19.7 | 0.87 | 73.13 |
| | 25% glass | | | | | |
| | fiber | | | | | |

^{[a]}Mw and Mn were measured by Gel permeation chromatography (GPC);
^{[b]}Glass transition temperature (T_{g}), melting point (Tₘ) and crystallinity were measured by DSC;
^{[c]}Onset and T_{d50%} and residue weight were characterized by TGA. Nylon 6 materials were subjected to drying overnight at 60° C. under vacuum to determine moisture content by the equation: (original mass of nylon 6-dried mass of nylon 6)/original mass of nylon 6*100%.

### Kinetic Modeling Methodology/Results

The following reaction network was used as a model for the calculation of the system's kinetic parameters:

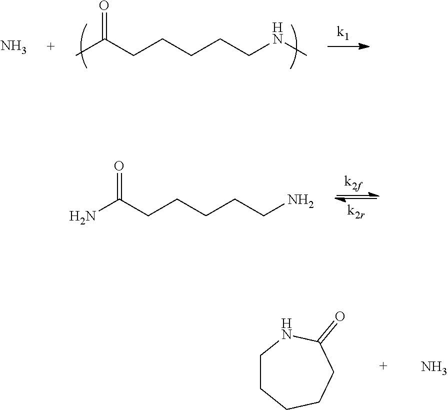

When first-order kinetics are assumed for each monomeric substrate the following governing differential equations result:

$$\frac{d {C}_{nylon - 6}}{d t} = - {k}_{1} {C}_{n y l o n - 6} {C}_{N H 3}$$

$$\frac{d {C}_{6 - aminohexanamide}}{d t} = {k}_{1} {C}_{n y l o n - 6} {C}_{N H 3} - {k}_{2 f} {C}_{6 - aminohexanamide} + {k}_{2 r} {C}_{caprolactam} {C}_{NH 3}$$

$$\frac{d {C}_{caprolactam}}{d t} = {k}_{2 f} {C}_{6 - aminohexanamide} - {k}_{2 r} {C}_{caprolactam} {C}_{NH 3}$$

Due to ammonia being approximately constant during reaction, we can lump its dependencies into the relevant rate constants (assuming the concentration to be approximately constant):

$$k {}_{n}^{′} = {k}_{n} {C}_{N H 3}$$

As such, we are left with the following:

$$\frac{d {C}_{n y l o n - 6}}{d t} = - k {}_{1}^{′} {C}_{n y l o n - {6}_{-} M E}$$

$$\frac{d {C}_{6 - aminohexanamide}}{d t} = k {}_{1}^{′} {C}_{n y l on - 6 \_ ME} - {k}_{2 f} {C}_{6 - aminohexanamide} + k {}_{2 r}^{′} {C}_{caprolactam}$$

$$\frac{d {C}_{caprolactam}}{d t} = {k}_{2 f} {C}_{6 - aminohexanamide} - k {}_{2 r}^{′} {C}_{caprolactam}$$

Where C_{nylon-6_ME} denotes the concentration of monomer equivalent for nylon-6 within the polymeric substrate, calculated assuming a molecular weight of 113.16 Da (0.353 M for 200 mg of nylon-6 in 5 mL of ethylene glycol). Once a kinetic model had been developed, it was fit to the experimental time series data below. For ease, molar concentrations were assumed (constant solution volume).

### Experimental Time Series Data

| Time (hrs) | C_{nylon-6} (M) | C_{6-aminohexanamide} (M) | C_{caprolactam} (M) |
|---|---|---|---|
| 0 | 0.353 | 0 | 0 |
| 0.5 | n.m. | 9.36E−04 | 0.00702 |
| 1.5 | n.m. | 0.00165 | 0.01406 |
| 3 | n.m. | 0.00547 | 0.03119 |
| 5 | n.m. | 0.01421 | 0.06452 |
| 7 | n.m. | 0.01609 | 0.09895 |
| 9 | n.m. | 0.02204 | 0.11947 |
| 15 | n.m. | 0.02176 | 0.16151 |
| 18 | n.m. | 0.03714 | 0.19547 |
| 24 | n.m. | 0.03789 | 0.23449 |

The MATLAB function fminunc( ) was then utilized to vary the kinetic rate constants for the differential equation system above while solving each iteration on the differential equation system using. It did so by minimizing the sum of the squared errors between the empirical data and the model output at each given time point. This resulted in the following kinetic rate constants which best fit the data.

### Kinetic Rate Constants

| Rate Constant (h⁻¹) | |
|---|---|
| k₁ | 0.0550 |
| k_{2f} | 2.6407 |
| k₂ᵣ | 0.3763 |

### Example 2: Further Synthesis of Exemplary Polymers

### Nylon 6 Materials

Nylon 6 powder and film were obtained from Goodfellow. Nylon pellets were purchased from Sigma-Aldrich. Fishing net, thread, T-shirt, carpet, and filament were purchased from Amazon. Washed and unwashed, contaminated nylon 6 fishing nets were obtained from Bureo.

### Chemicals and Solvents

Propyl 6-aminohexanoate hydrochloride was purchased from Chemspace. Methanol (99.9%) was purchased from VWR chemicals. Anhydrous ethanol was purchased from Koptec. Hexane (AR/ACS) was purchased from Macron Fine Chemicals. Sodium hydride (60% dispersion in mineral oil), ε-Caprolactam (99%), 1-Bromopropane (99.9%), propanol (≥99.5%), isopropanol (≥99.5%), ethylene glycol (≥99%), acetone (≥99.5%) and ethyl acetate (≥99.5%) were purchased from Sigma-Aldrich. 1,3,5-tri-tert-butylbenzene (>98.0%) was purchased from TCI.

### Catalysts

H₃PO₄ (≥85 wt % in water) aqueous solution, (NH₄)₂HPO₄ (≥98%), and (NH₄)₂SO₄ (≥99.5%) were purchased from Sigma-Aldrich. Lanthanum (III) trifluoromethanesulfonate (99%), tin (II) trifluoromethanesulfonate (98%), and cerium (IV) trifluoromethanesulfonate (98%) were purchased from Fisher Scientific.

### General Experimental Procedures

The alcohol-assisted depolymerization of nylon 6 materials in presence of a H₃PO₄ catalyst was carried out in a 25 mL high-pressure Parr autoclave, equipped with a Teflon magnetic stirring bar. Typically, the autoclave was loaded with 200 mg nylon 6 substrate, 0.2 mmol H₃PO₄, and 5 mL propanol. The reactor was then sealed and flushed with N₂ for 3 times. The reactor was heated between 190-220° C. and stirred at 400 rpm for the duration of the reaction. After the completion of the reaction, the reactor was immediately quenched in an ice bath. When the temperature of the reactor stabilized at room temperature, it was then neutralized with ammonia gas. After this, 40 mL methanol and approximately 30 mg of a 1,3,5-tri-tert-butylbenzene (TTB) external standard (weighed) were then added to the crude depolymerized mixture. The suspension was then vigorously shaken and sonicated to ensure complete solubilization of formed reaction products. The suspension was then subjected to centrifugation to separate the liquids and methanol insoluble fractions prior to characterization.

### Product Identification and Quantification Via Gas Chromatography

The liquid products were then identified via gas chromatography coupled with mass spectrometry (GC-MS) and quantified via gas chromatography with flame ionization detection (GC-FID) based on rigorous calibration curves against a 1,3,5-tri-tert-butylbenzene external standard. The GC-FID system comprised an Agilent 7890A equipped with a 7693 autosampler and an HP-5MS (30 m×250 μm×0.25 μm) capillary column. The method consisted of a 1 μL injection volume, a 10:1 split ratio, an inlet temperature of 280° C., and a hydrogen carrier gas. For each run, the temperature was initially held for 1 min at 50° C., followed by a ramp to 280° C. (10° C.·min⁻¹) and a hold for 9 min prior to method termination. Meanwhile, the GC-MS consisted of an Agilent 7820A system, a 5977B single quadrupole MS detector, and an identical column to the GC-FID system. The utilized GC-MS method was identical to that on the GC-FID but instead used a helium carrier gas.

Preparation and Boiling Point Measurement of 1-propylazepan-2-one

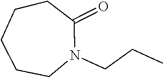

In a typical procedure, to a stirred solution of ε-caprolactam (1.13 g, 10 mmol) in anhydrous THF (30 mL) at 25° C., NaH (60% suspension in mineral oil, 12 mmol, 0.48 g) was proportionally added. The reaction mixture was stirred for 30 min, followed by the dropwise addition of 1-bromopropane (12 mmol, 1.23 g). The resulting mixture was then heated and refluxed at 80° C. for 2 h, cooled to room temperature, and quenched with water (20 mL). The THF solvent was subsequently removed in vacuo and the remaining residue was extracted with EtOAc (3×50 mL). The combined organic layers were dried over anhydrous MgSO₄, filtered, and concentrated in vacuo to afford the crude reaction mixture. Finally, the product (1-propylazepan-2-one) was isolated by silica gel column chromatography (gradient elution: EtOAc and hexane—50/50). 1-propylazepan-2-one (isolated yield: 52.9%), transparent liquid. ¹H NMR (400 Mz, CDCl₃) δ 3.3-3.25 (m, 4H), 2.48-2.45 (m, 2H), 1.68-1.57 (m, 6H), 1.51-1.44 (m, 2H), 0.87-0.82 (m, 3H); ¹³C NMR (126 MHz, CDCl₃) δ 175.78, 50.00, 49.70, 37.43, 30.14, 28.82, 23.61, 21.41, 11.46.

The boiling point of 1-propylazepan-2-one (251.8° C.) was determined by DSC-TGA conducted with 10 mg of synthesized compound loaded in a hermetically sealed pan with a pinhole lid. The instrument consisted of a SDT Q600 Analyzer (TA Instruments). The method utilized a nitrogen purge gas at 50 mL·min⁻¹ and a heating ramp of 10° C.·min⁻¹. The boiling point was defined as the minima in the measured heat flow.

### Spectroscopic Characterizations

Gel permeation chromatography (GPC) was performed to determine polymer absolute weight-average molar mass (M_{w}), number-average molecular weight (Mₙ), and molecular weight dispersity (Ð=M_{w}/Mₙ). The instrument consisted of an Agilent HPLC system equipped with one guard column and two PLgel 5 μm mixed-C gel permeation columns and coupled with a Wyatt DAWN HELEOS II multi (18)-angle light scattering detector and a Wyatt Optilab TrEX dRI detector. The analysis was performed at 70° C. using N,N-dimethylformamide (DMF) as the eluent at a flow rate of 1.0 mL/min. Wyatt ASTRA 7.1.2 molecular weight characterization software was used for analysis.

Thermogravimetric analysis (TGA) was performed at Colorado State University on a Q50 TGA Analyzer (TA Instrument) and at the Massachusetts Institute of Technology on a Q500 TGA Analyzer (TA Instruments) to determine polymer decomposition temperatures. Typically, 1-3 mg of sample was initially weighed into a tared platinum pan. The sample was then heated from 20 to 600° C. at a heating rate of 10° C.·min⁻¹ under a 20 mL·min⁻¹ flow of nitrogen. The decomposition temperatures were recorded as the point at which 5% mass loss (T_{d,5%}) occurred.

Differential scanning calorimetry (DSC) was conducted on an Auto Q20 (TA Instruments) to determine glass transition temperatures (T_{g}). All Tₘ and T_{g} values were obtained from the second scan unless otherwise indicated. Both heating rate and cooling rate were 10° C./min unless otherwise indicated. In a typical procedure, the sample (5-10 mg) was weighed into an aluminum DSC pan and capped. The sample was sealed and heated from 25 to 250° C. at a heating rate of 10° C.·min⁻¹. It was then cooled to 25° C. at a rate of 10° C.·min⁻¹. Subsequently, a second heating scan to 250° C. at the same rate was performed. All the experiments were performed under a 20 mL·min⁻¹ nitrogen flow.

### Product Quantification

Product yield was obtained by GC-FID based on calibration curves using a 1,3,5-tri-tert-butylbenzene external standard, using the following formula:

$$Carbon molar yield ( \% ) = \frac{Molar carbon of product obtained experimentally}{Theorectial molar total carbon of nylon 6} \times 100 \%$$

### Catalyst Screening for the Deconstruction of Nylon 6 to ε-Caprolactam^{[a]}

| | | Carbon yield^{[b]} (mol %) | | | | |
|---|---|---|---|---|---|---|
| En- try | Catalyst | 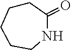 | 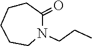 | 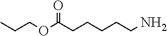 | 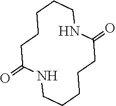 [SMILES: O=C1CCCCCNC(=O)CCCCCN1] | 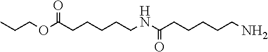 [SMILES: CCCOC(=O)CCCCCNC(=O)CCCCCN] |
| 1 | None | 2.10 | 0.01 | 0.13 | 0.07 | 0.01 |
| 2 | HCl | 10.58 | 0.22 | 1.05 | 0.29 | 1.29 |
| 3 | H₂SO₄ | 45.09 | 0.23 | 9.33 | 3.48 | 0.79 |
| 4 | H₃PO₄ | 74.35 | 0.74 | 6.43 | 1.11 | 4.89 |
| 5 | (NH₄)₂HPO₄ | 61.22 | 0.86 | 6.24 | 0.83 | 3.24 |
| 6 | Sn(OTf)₂ | 39.97 | 0.08 | 5.65 | 4.74 | 0.17 |
| 7 | Ce(OTf)₄ | 31.90 | 0.06 | 12.66 | 2.57 | 3.31 |
| 8 | La(OTf)₃ | 23.16 | 0.00 | 1.71 | 0.26 | 0.69 |

[a] Reaction conditions: 200 mg nylon 6 pellet, 0.2 mmol catalyst (HCI: 0.6 mmol, H₂SO₄: 0.3 mmol), 5 mL n-PrOH, 220° C, 2 h. [b] Yields were quantified by GC-FID based on calibration curves using a 1,3,5-tri-tert-butylbenzene external standard.

### Solvent Screening for the Deconstruction of Nylon 6 to ε-Caprolactam^{[a]}

| | | Carbon yield^{[b]} (mol %) | | | | |
|---|---|---|---|---|---|---|
| En- try | Solvent | 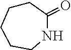 | 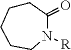 | 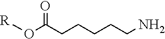 | 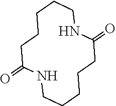 [SMILES: O=C1CCCCCNC(=O)CCCCCN1] | 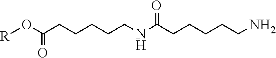 |
| 1 | MeOH | 44.51 | 5.22 | 18.33 | 1.24 | 7.23 |
| 2 | EtOH | 37.94 | 1.08 | 5.16 | 1.31 | 3.85 |
| 3 | n-PrOH | 74.35 | 0.74 | 6.43 | 1.11 | 4.89 |
| 5 | iPrOH | 27.96 | 0.00 | 1.23 | 0.00 | 0.00 |
| 6 | n-BuOH | 77.19 | 0.00 | 4.82 | 1.35 | 2.91 |
| 7 | EG | 23.34 | 0.00 | 0.00 | 0.00 | 0.00 |

[a] Reaction conditions: 200 mg nylon 6 pellet, 0.2 mmol H₃PO₄, 5 mL solvent, 220° , 2 h; R=methyl, ethyl, or propyl group; [b] Yields were quantified by GC-FID based on calibration curves using a 1,3,5-tri-tert-butylbenzene external standard.

### Temperature Screening for the Deconstruction of Nylon 6 to ε-Caprolactam^{[a]}

| | | Carbon yield^{[b]} (mol %) | | | | |
|---|---|---|---|---|---|---|
| En- try | T (° C.) | 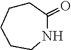 | 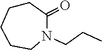 | 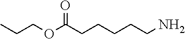 | 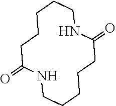 [SMILES: O=C1CCCCCNC(=O)CCCCCN1] | 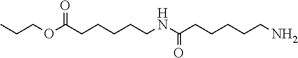 [SMILES: CCCOC(=O)CCCCCNC(=O)CCCCCN] |
| 1 | 190 | 29.92 | 0.01 | 3.99 | 2.50 | 3.86 |
| 2 | 200 | 54.95 | 0.28 | 6.74 | 1.96 | 3.03 |
| 3 | 210 | 71.24 | 0.24 | 7.99 | 1.67 | 4.74 |
| 4 | 220 | 74.35 | 0.74 | 6.43 | 1.11 | 4.89 |

[a] Reaction conditions: 200 mg nylon 6 pellet, 0.2 mmol H₃PO₄, 190-220° C., 5 mL n-PrOH, 2 h. [b] Yields were quantified by GC-FID based on calibration curves using a 1,3,5-tri-tert-butylbenzene external standard.

### Reaction Time Screening for the Deconstruction of Nylon 6 to ε-Caprolactam^{[a]}

| | | Carbon yield^{[b]} (mol %) | | | | |
|---|---|---|---|---|---|---|
| En- try | Time (h) | 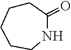 | 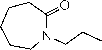 | 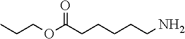 | 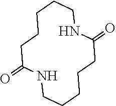 [SMILES: O=C1CCCCCNC(=O)CCCCCN1] | 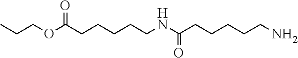 [SMILES: CCCOC(=O)CCCCCNC(=O)CCCCCN] |
| 1 | 0.25 | 6.33 | 0.00 | 1.55 | 0.20 | 1.93 |
| 2 | 0.50 | 14.66 | 0.00 | 2.96 | 0.51 | 3.61 |
| 3 | 0.75 | 21.87 | 0.00 | 3.55 | 0.36 | 4.01 |
| 4 | 1.00 | 33.01 | 0.00 | 4.80 | 0.44 | 4.56 |
| 5 | 1.5 | 43.50 | 0.03 | 5.86 | 0.69 | 5.74 |
| 6 | 2 | 54.95 | 0.30 | 7.17 | 2.08 | 3.23 |
| 7 | 3 | 63.58 | 0.48 | 7.61 | 0.94 | 4.86 |
| 8 | 4 | 73.12 | 0.16 | 8.30 | 0.48 | 3.03 |
| 9 | 5 | 73.61 | 0.30 | 8.83 | 1.06 | 4.40 |
| 10 | 6 | 74.85 | 0.49 | 8.90 | 1.17 | 4.33 |

[a] Reaction conditions: 200 mg nylon 6 pellet, 0.2 mmol H₃PO₄, 200° C., 5 mL n-PrOH, 0.25-6 h; [b] Yields were quantified by GC-FID based on calibration curves using a 1,3,5-tri-tert-butylbenzene external standard.

### Nylon 6 Loading for the Deconstruction of Nylon 6 to ε-Caprolactam^{[a]}

| | | Carbon yield^{[b]} (mol %) | | | | |
|---|---|---|---|---|---|---|
| En- try | load- ing (mg) | 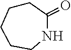 | 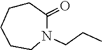 | 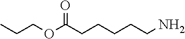 | 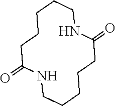 [SMILES: O=C1CCCCCNC(=O)CCCCCN1] | 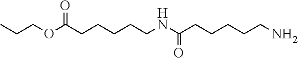 [SMILES: CCCOC(=O)CCCCCNC(=O)CCCCCN] |
| 1 | 100 | 29.92 | 0.01 | 3.99 | 2.50 | 3.86 |
| 2 | 200 | 54.95 | 0.28 | 6.74 | 1.96 | 3.03 |
| 3 | 400 | 71.24 | 0.24 | 7.99 | 1.67 | 4.74 |
| 4 | 600 | 74.35 | 0.74 | 6.43 | 1.11 | 4.89 |

[a] Reaction conditions: 100-600 mg nylon 6 pellet, 0.2 mmol H₃PO₄, 210° C., 5 mL n-PrOH, 2.5 h; [b] Yields were quantified by GC-FID based on calibration curves using a 1,3,5-tri-tert-butylbenzene external standard.

### Catalyst Loading Screening for the Deconstruction of Nylon 6 to ε-Caprolactam^{[a]}

| | | Carbon yield^{[b]} (mol %) | | | | |
|---|---|---|---|---|---|---|
| En- try | Catalyst loading (wt %) | 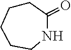 | 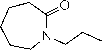 | 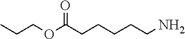 | 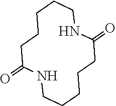 [SMILES: O=C1CCCCCNC(=O)CCCCCN1] | 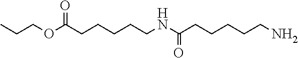 [SMILES: CCCOC(=O)CCCCCNC(=O)CCCCCN] |
| 1 | 5 | 59.21 | 5.24 | 0.11 | 0.60 | 3.17 |
| 2 | 10 | 74.04 | 7.79 | 0.31 | 1.01 | 3.60 |
| 3 | 20 | 71.21 | 14.39 | 0.70 | 1.19 | 6.14 |

[a] Reaction conditions: 200 mg nylon 6 pellet, 5-20 wt% H₃PO₄, 210° C., 2.5 h, 5 mL n-PrOH; [b] Yields were quantified by GC-FID based on calibration curves using a 1,3,5-tri-tert-butylbenzene external standard.

### The Deconstruction of Different Nylon 6 Feedstock to ε-Caprolactam^{[a]}

| | | Carbon yield^{[d]} (mol %) | | | | |
|---|---|---|---|---|---|---|
| En- try | Nylon feed- stock | 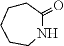 | 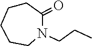 | 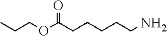 | 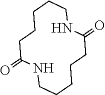 [SMILES: O=C1CCCCCNC(=O)CCCCCN1] | 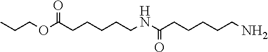 [SMILES: CCCOC(=O)CCCCCNC(=O)CCCCCN] |
| 1 | Powder | 68.91 | 0.39 | 8.45 | 1.48 | 3.29 |
| 2 | Film | 71.62 | 0.00 | 8.90 | 1.15 | 4.83 |
| 3 | Pellet | 74.04 | 0.31 | 7.79 | 1.01 | 3.60 |
| 4 | Fishing | 70.73 | 0.34 | 8.45 | 1.02 | 4.42 |
| | net | | | | | |
| | (green) | | | | | |
| 5 | Fishing | 76.44 | 0.33 | 9.69 | 1.35 | 5.49 |
| | net | | | | | |
| | (white) | | | | | |
| 6 | Fishing | 74.52 | 0.38 | 9.23 | 1.00 | 4.67 |
| | net | | | | | |
| | (mixture) | | | | | |
| 7 | Thread | 74.91 | 0.37 | 8.79 | 1.03 | 4.12 |
| 8 | T-shirt | 69.23 | 0.21 | 9.99 | 1.04 | 4.33 |
| 9 | carpet | 73.18 | 0.43 | 9.91 | 0.96 | 4.05 |
| 10 | filament | 70.28 | 0.53 | 7.03 | 0.95 | 3.67 |
| 12 | Washed | 70.16 | 0.37 | 8.60 | 0.89 | 4.12 |
| | fishing | | | | | |
| | net | | | | | |
| 13 | Un- | 67.46 | 0.33 | 8.65 | 0.83 | 3.58 |
| | washed | | | | | |
| | fishing | | | | | |
| | net^{[b]} | | | | | |
| 14 | Multi- | 68.75 | 1.19 | 17.05 | 0.70 | 1.35 |
| | layer | | | | | |
| | film^{[c]} | | | | | |

[a] Reaction conditions: 200 mg nylon 6 feedstock, 0.2 mmol H₃PO₄, 210° C., 5 mL n-PrOH, 2.5 h. [b] Unwashed fishing net may contain sea salt, sand and other unknown impurities. [c] Multilayer film contains nylon 6 (25 wt%), LDPE (15 wt%), LLDPE (17 wt%), VLDPE (12 wt%), LLTIE (4 wt%), EVOH (5 wt%), LLTIE (5 wt%) and VLDPE (16 wt%); LDPE: low-density of polyethylene; LLDPE: linear low-density polyethylene; VLDPE: very low-density of polyethylene; LLTIE: linear low density polyethylene graft maleic anhydride; EVOH: ethylene vinyl alcohol. [d] Yields were quantified by GC-FID based on calibration curves using a 1,3,5-tri-tert-butylbenzene external standard.

### Characterization Results for Commercial Nylon 6 Materials

| Nylon 6 | | Mw^{[a]} | Mn^{[a]} | T_{g}^{[b]} | Tₘ^{[b]} | Onset^{[c]} |
|---|---|---|---|---|---|---|
| materials | Compositions | (kDa) | (kDa) | (° C.) | (° C.) | (° C.) |
| Powder | Nylon 6 | 23.5 | 9.9 | 48 | 220 | 417 |
| Film | Nylon 6 | 38.1 | 20.6 | 65 | 226 | 437 |
| Pellet | Nylon 6 | 39.9 | 26.7 | 51 | 224 | 427 |
| Fishing net | Nylon 6 | 32.5 | 21.0 | 53 | 224 | 429 |
| (Green) | | | | | | |
| Fishing net | Nylon 6 | 29.9 | 20.6 | 53 | 223 | 425 |
| (white) | | | | | | |
| Thread | Nylon 6 | 40.0 | 28.2 | 52 | 220 | 427 |
| T-shirt | 97% nylon 6 | 44.3 | 26.7 | 53.2 | 225 | 421 |
| | 3% spandex | | | | | |
| Carpet | nylon 6 | 31.9 | 22.5 | 51.6 | 223 | 421 |
| Filament | 75% nylon 6 | N.D. | N.D. | N.D. | 219 | 422 |
| | 25% glass fiber | | | | | |
| Washed fishing | Nylon 6 | 38.26 | 30.37 | 56.3 | 220 | 431 |
| net | | | | | | |
| Unwashed | Nylon 6 | 41.72 | 33.39 | 61.2 | 212 | 426 |
| fishing net^{[d]} | | | | | | |

| | | | | Moisture | Nylon 6 |
|---|---|---|---|---|---|
| Nylon 6 | T_{d, 50%}^{[c]} | Residue^{[c]} | Crystallinity^{[b]} | content | Content |
| materials | (° C.) | (%) | (%) | (%) | (%) |
| Powder | 447 | 2.6 | 35.8 | 2.50 | 94.9 |
| Film | 467 | 0.1 | 22.1 | 1.86 | 98.04 |
| Pellet | 459 | 0 | 28.2 | 0.44 | 99.56 |
| Fishing net | 457 | 0.1 | 32.9 | 3.35 | 96.55 |
| (Green) | | | | | |
| Fishing net | 455 | 0 | 32.1 | 3.2 | 96.8 |
| (white) | | | | | |
| Thread | 455 | 0 | 32.5 | 2.59 | 97.41 |
| T-shirt | 455 | 2.7 | N.D. | 1.8 | 95.5 |
| Carpet | 455 | 1.5 | 28.8 | 3.04 | 95.46 |
| Filament | 456 | 26.0 | 19.7 | 0.87 | 73.13 |
| Washed fishing | 465 | 4.6 | 18.2 | 1.43 | 93.97 |
| net | | | | | |
| Unwashed | 469 | 3.4 | 20.3 | 1.54 | 95.06 |
| fishing net^{[d]} | | | | | |

^{[a]}M_{w} and Mₙ were measured by gel permeation chromatography (GPC);
^{[b]}Glass transition temperature (T_{g}), melting point (Tₘ) and crystallinity were measured by DSC;
^{[c]}Onset and T_{d50%} and residue weight were characterized by TGA.
^{[d]}Unwashed fishing net may contain sea salt, sand and other unknown impurities.

### Kinetic Modeling Methodology/Results

The following simplified reaction network was used as a model for the calculation of the system's kinetic parameters:

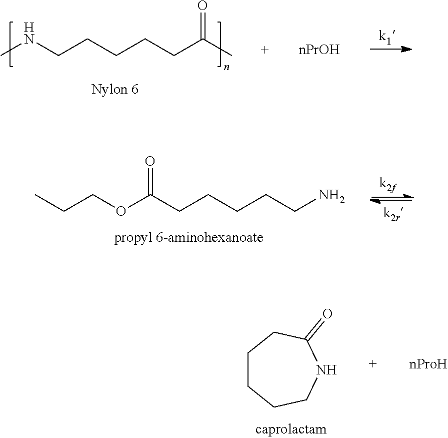

When first-order kinetics are assumed for each monomeric substrate the following governing differential equations result:

$$\frac{d {C}_{n y l o n - 6}}{d t} = - {k}_{1} {C}_{n y l o n - 6} {C}_{n - P r O H}$$

$$\frac{d {C}_{propyl 6 - aminohexanoate}}{d t} = {k}_{1} {C}_{n y l o n - 6} {C}_{n - P r O H} - {k}_{2 f} {C}_{propyl 6 - aminohexanoate} + {k}_{2 r} {C}_{caprolactam} {C}_{n - PrOH}$$

$$\frac{d {C}_{caprolactam}}{d t} = {k}_{2 f} {C}_{propyl 6 - aminohexanoate} - {k}_{2 r} {C}_{{caprolactamC}_{n - PrOH}}$$

Where C_{nylon-6} denotes the concentration of monomer equivalent for nylon 6 within the polymeric substrate, calculated assuming a molecular weight of 113.16 Da (0.353 M for 200 mg of nylon 6 in 5 mL of ethylene glycol).

Due to the large excess of n-PrOH present during reaction, we can assume its concentration as approximately constant during reaction, and thereby lump its dependencies into the relevant rate constants:

$${k}_{n}^{′} = {k}_{n} {C}_{n - PrOH}$$

As a result, we are left with the following:

$$\frac{d {C}_{n y l o n - 6}}{d t} = - {k}_{1}^{′} {C}_{n y l o n - 6}$$

$$\frac{d {C}_{propyl 6 - aminohexanoate}}{d t} = {k}_{1}^{′} {C}_{n y l o n - 6} - {k}_{2 f} {C}_{propyl 6 - aminohexanoate} + {k}_{2 r}^{′} {C}_{c a p r o l a c t a m}$$

$$\frac{d {C}_{caprolactam}}{d t} = {k}_{2 f} {C}_{propyl 6 - aminohexanoate} - {k}_{2 r}^{′} {C}_{c a p r o l a c t a m}$$

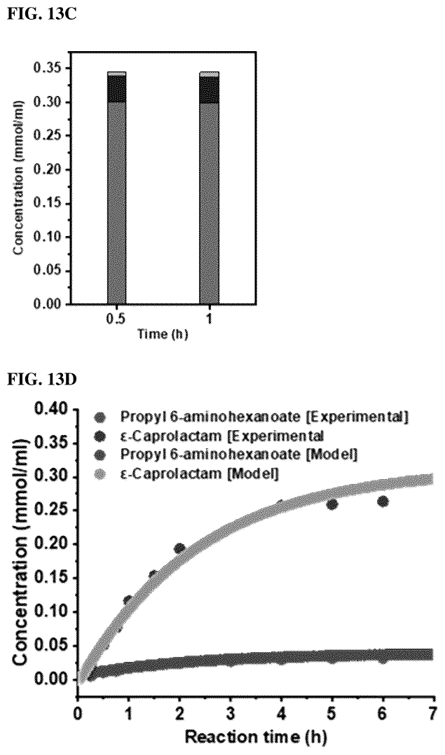

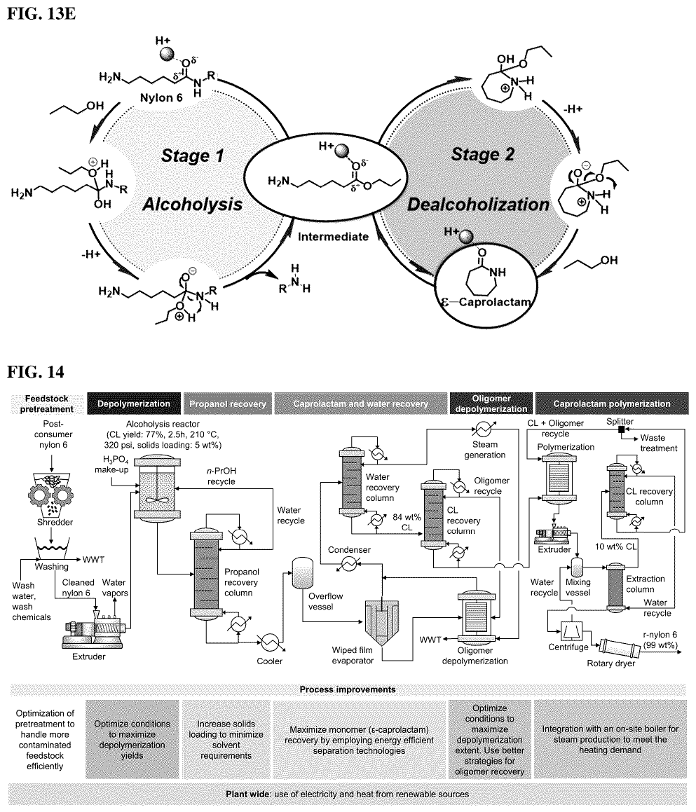

Finally, we can further simplify our system, and eliminate a free variable, by incorporating the equilibrium constant between propyl 6-aminohexanoate and caprolactam as defined by concentrations as reported in FIG. 13C of the main text:

$${K}_{2}^{e q} = \frac{{k}_{2 f}}{{k}_{2 r}^{′}} = \frac{[ caprolactam ]}{[ propyl 6 - a min o h e x a n o a t e ]} = 7.988$$

Which results in the following system of governing equations:

$$\frac{d {C}_{nylon - 6}}{d t} = - {k}_{1}^{′} {C}_{nylon - 6}$$

$$\frac{d {C}_{propyl 6 - aminohexanoate}}{d t} = {k}_{1}^{′} {C}_{nylon - 6} - {k}_{2 f} {C}_{propyl 6 - aminohexanoate} + \frac{{k}_{2 f}}{{K}_{2}^{e q}} {C}_{caprolactam}$$

$$\frac{d {C}_{caprolactam}}{d t} = {k}_{2 f} {C}_{propyl 6 - aminohexanoate} - \frac{{k}_{2 f}}{{K}_{2}^{e q}} {C}_{caprolactam}$$

Once a kinetic model had been developed, it was fit to the experimental time series data below. For ease, molar concentrations were assumed (constant solution volume).

| Time | C_{nylon-6} | C_{propyl 6-aminohexanoate} | C_{caprolactam} |
|---|---|---|---|
| (h) | (M) | (M) | (M) |
| 0 | 0.353 | — | — |
| 0.25 | n.m. | 0.00546 | 0.02232 |
| 0.5 | n.m. | 0.01044 | 0.05168 |
| 0.75 | n.m. | 0.01253 | 0.07707 |
| 1 | n.m. | 0.01691 | 0.11632 |
| 1.5 | n.m. | 0.02066 | 0.15332 |
| 2 | n.m. | 0.02374 | 0.19366 |
| 3 | n.m. | 0.02681 | 0.22406 |
| 4 | n.m. | 0.02946 | 0.25769 |
| 5 | n.m. | 0.03111 | 0.25942 |
| 6 | n.m. | 0.03136 | 0.26379 |

MATLAB was then utilized to vary the kinetic rate constants for the differential equation system above while solving each iteration on the differential equation system. It did so by minimizing the sum of the squared errors between the empirical data and the model output at each given time point. A nominal initial guess of

$$[ {k}_{1}^{′} , {k}_{2 f} ] = [ 1 , 1 ] {h}^{- 1}$$

was utilized. This resulted in the following kinetic rate constants which best fit the data:

| Rate Constant (h⁻¹) | |
|---|---|
| ${k}_{1}^{′}$ | 0.423 |
| k_{2f} | 29.6 |
| ${k}_{2 r}^{′} ( Calculated via {K}_{2}^{eq} )$ | 3.71 |

### Results and Discussion

### Establishing the Optimized Reaction Conditions for Nylon 6 Depolymerization

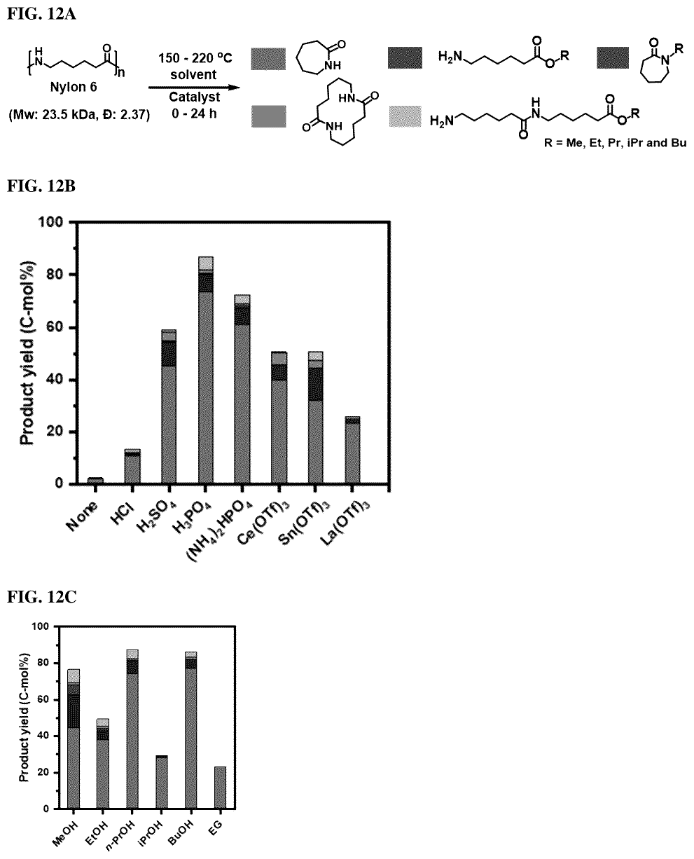

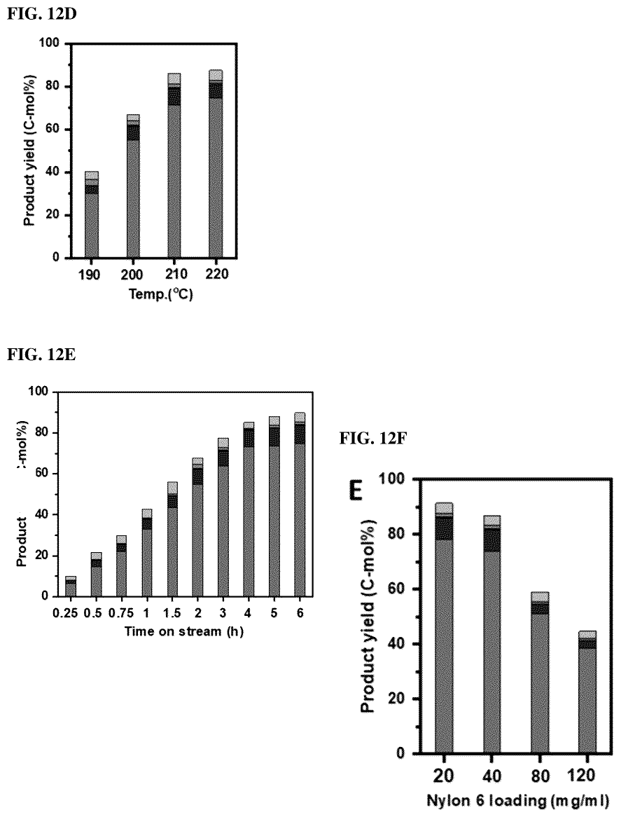

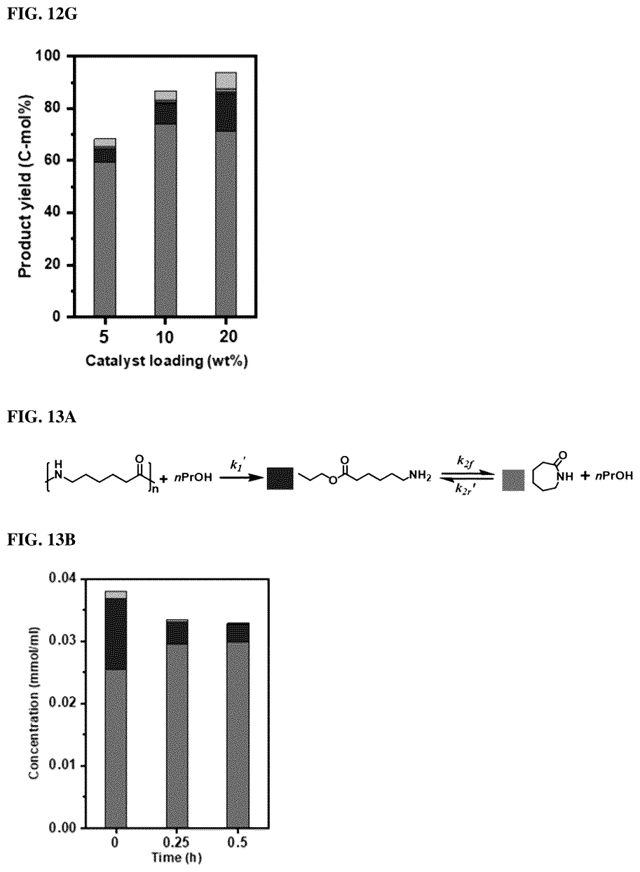

We initially hypothesized that an acid catalyst could serve as an active center for both alcoholysis and cyclodealcoholization by coordinating with carbonyl oxygens, thereby enhancing their susceptibility to nucleophilic attack by alcohols. To this end, a range of different acid catalysts, including both heterogeneous and homogeneous Lewis and Brønsted acids, were initially screened in an n-PrOH solvent for the depolymerization of nylon 6 at 220° C. [FIG. 12B]. Uncatalyzed alcoholysis of nylon 6 was ineffective, consistent with previous reports. However, in the presence of Brønsted acids (HCl, H₂SO₄ and H₃PO₄), the yield of ε-caprolactam was improved to 10.6, 45.1, 74.4 mol % respectively, implying that the presence of an acid catalyzes alcoholysis of nylon 6 while also potentially aiding subsequent cyclodealcoholization. Interestingly, the various acids demonstrated substantial difference in nylon 6 alcoholysis, suggesting potential differences with respect to either acid solubilities or dissociation constants in n-PrOH. Aside from ε-caprolactam, alcoholysis also produces propyl 6-aminohexanoate as well as other dimeric intermediates, namely 1,8-diazacyclotetradecane-2,9-dione and propyl 6-(6-Aminohexanamido)hexanoate. The use of a phosphate salt [(NH₄)₂HPO₄] likewise resulted in ε-caprolactam generation at a yield of 61%, confirming NH₄⁺ ions, which can also act as Brønsted acids, are capable of coordinating carbonyl groups and thereby promoting nucleophilic attack by n-PrOH. In contrast, homogeneous Lewis acid catalysts such as Sn(OTf)₃, Ce(OTf)₄, and La(OTf)₃ afforded lower ε-caprolactam yields, ranging between 20-40 mol %.

Based on the high resulting ε-caprolactam yields when combining H₃PO₄ and n-PrOH, we elected to further investigate the influence of solvents on reaction performance [FIG. 12C]. Volatile alcohols were first examined owing to their low cost and ease of downstream separation. Methanol (MeOH) led to a moderate yield of 44.5 mol %, higher than that achieved using ethanol (EtOH) (34.9 mol %). These results were inferior compared with those for larger protic alcohols, where n-PrOH and n-butanol (n-BuOH) achieved yields of 74.3 and 77.2 mol %, respectively. This is in line with previous observations where under supercritical reaction conditions, higher yields of ε-caprolactam typically obtained in those larger alcohols from the depolymerization of nylon 6 via alcoholysis. On the other hand, isopropanol (iPrOH) resulted in a decreased yield of 28.0 mol %, only slightly higher than that obtained with ethylene glycol (EG) (23.3 mol %). These observed differences in reactivity could be related to differences in solvent-substrate interactions (e.g., hydrogen bonding donating or accepting propensity), which could potentially enhance/demote the electrophilicity of carbonyl groups or the nucleophilicity of utilized alcohols, thereby modifying the efficacy of a given system for the cleavage of amide bonds in nylon 6.

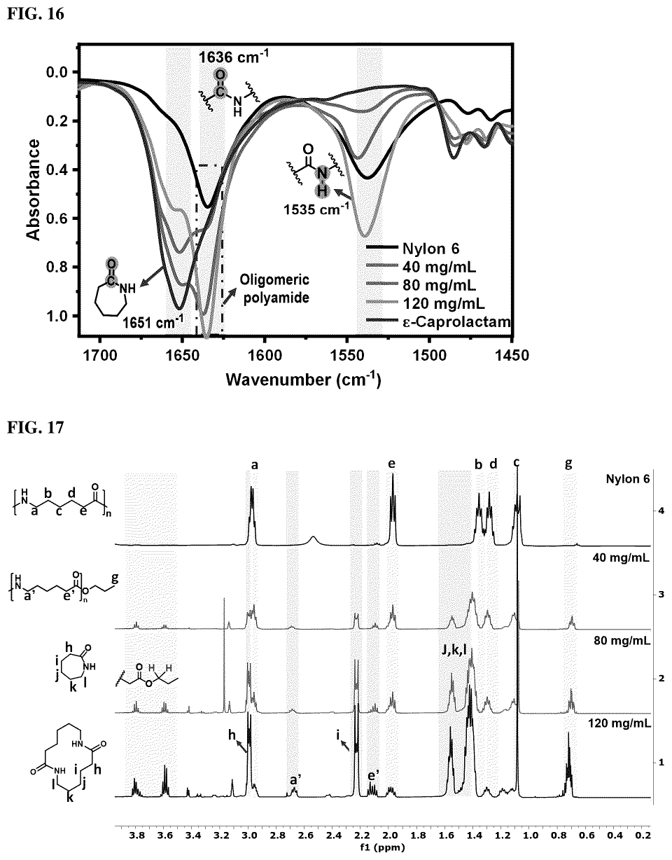

Moving forward with n-PrOH as a viable, high-performing solvent medium, multiple depolymerization temperatures (190, 200, 210 and 220° C.) were investigated [FIG. 12D]. ε-Caprolactam yields generally increased with increasing reaction temperature, ranging from ˜30 mol % at 190° C. to 74.4 mol % at 220° C. The influence of reaction time was also investigated at an intermediate reaction temperature (200° C.) [FIG. 12E]. The production of ε-caprolactam was significantly favored at longer reaction times, reaching a yield of 74.8 mol % after 6 h. Interestingly, this increase was coupled with a slight increase in the production of propyl 6-aminohexanoate. Next, owing to the effects reactor sizing can have on process economics, the effect of substrate loading was examined. Increased nylon 6 loadings resulted in decreased yields of ε-caprolactam, ranging from 78.3 mol % at a loading of 20 mg/mL to 38.7 mol % at 120 mg/mL [FIG. 12F]. This decline in depolymerization extent at high solids loading likely stems from the decreasing catalyst/substrate ratio, which is supported by both FTIR and ¹H-NMR of the crude reaction residues which confirmed the presence of unconverted oligomeric polyamide fragments [FIGS. 16 & 17]. In turn, the influence of catalyst loading on nylon 6 alcoholysis was also investigated. Unsurprisingly, increasing the catalyst loading from 5 to 10 wt % H₃PO₄ enhanced the ε-caprolactam yield from 59.2 to 74.0 mol %. However, further increasing the concentration to 20 wt % H₃PO₄ resulted in a decreased ε-caprolactam yield of 71.2 mol % while instead promoting the production of propyl 6-aminohexanoate [FIG. 12G].

Mechanistic Insights into the Depolymerization of Nylon 6

To elucidate the reaction mechanism and kinetics for the depolymerization of nylon 6, a series of probe reactions on model substrates was conducted. It was initially postulated that the reaction likely occurs predominantly via a propyl 6-aminohexanoate intermediate [FIG. 13A], as the compound is seen as a side product in many of the depolymerization reactions described in FIGS. 12A-12G. This was verified using a purified model propyl 6-aminohexanoate, which underwent rapid, near-complete cyclodealcoholization (90 mol % conversion) to ε-caprolactam within a 15-minute reaction period under similar conditions to those shown in FIG. 13B. Further increasing the reaction time led to no significant variation in the reagent and product concentrations, implying a potential equilibrium between propyl 6-aminohexanoate and ε-caprolactam. This was further supported using pure ε-caprolactam as a probe, which was indeed converted back into propyl 6-aminohexanoate under similar conditions [FIG. 13C].

Based upon the product distributions and model results, a plausible acid-catalyzed reaction mechanism for the alcoholysis of nylon 6 involving consecutive alcoholysis and cyclodealcoholization steps is proposed, as described in FIG. 13E. In Stage 1, the amide bond of nylon 6 is initially coordinated and activated by a Brønsted acid, making it more susceptible to nucleophilic attack by n-PrOH to produce propyl 6-aminohexanoate. This can then undergo cyclodealcoholization during Stage 2 to yield ε-caprolactam, with n-PrOH being regenerated in the reaction.

To better understand relative reaction rates for the system, a simple first-order, pseudo-homogeneous kinetic model as developed utilizing the network outlined in FIG. 13A. For ease of calculation, and owing to the large excess of n-PrOH in the system, we assumed alcohol concentration in the liquid phase to be approximately constant, and thereby lumped the effects of n-PrOH concentration into the apparent kinetic rate constants [k′ₙ=kₙC_{n-PrOH}]. The ratio of [K₂^{eq}=k_{2f}/k′₂ᵣ=8.0] was assumed to be constant as defined by the equilibrium concentrations shown in FIG. 13C. Finally, we further simplified the system by assuming the equilibrium between nylon 6 depolymerization and repolymerization was negligible.

Using this simplified system, experimental concentrations of ε-caprolactam and propyl 6-aminohexanoate were fit to a first-order power law model by optimization of the first-order kinetic constants: k′₁ and k_{2f} [FIG. 13D]. A sum-squared-error cost function was utilized by assuming an initial molar nylon concentration equivalent to the molar concentration of monomeric subunits within the nylon polymer. The kinetic constant k′₁ was thereby determined to be 0.423 h⁻¹. However, k_{2f} demonstrated initial guess sensitivity. As a result, a sensitivity analysis was implemented holding k′₁ constant, determining the parameters k_{2f} and k′₂ᵣ to be >20 and >2.5 h⁻¹, respectively. These results reinforce experimental observations suggesting release of monomers from nylon 6 via alcoholysis proceeds slower than cyclodealcoholization.

### Depolymerization of Commercial Nylon Feedstocks to ε-Caprolactam

Following our reaction optimization and mechanistic investigation, we expanded our catalytic methodology to commercial nylon 6 plastic wastes including commercial fishing nets (green and white), 3D printing filament, film, t-shirt fabric, carpet, and thread, all of which were chopped into small pieces (˜1 mm in length) prior to being loaded into reactors for subsequent depolymerization. Nylon 6 powder and film both delivered yields of approximately 70 mol % to ε-caprolactam under optimized reaction conditions [FIG. 7]. Meanwhile, the depolymerization of fishing net (green, white, or a mixture of both), a significant contributor to ocean plastic waste pollution, achieved the highest yield (upwards of 76 mol %). Apart from demonstrating that our process works on commercial polymers, this result also suggests that this chemistry is resilient to dyes and other additives found in commercial nylon 6 materials.

We also applied the system to carpet fibers, which have an estimated annual production of 12 billion ft², 91% of which ends up in landfills at end-of-life. Under our standard reaction conditions, the carpet was converted to ε-caprolactam at a yield of 73.2 mol %. Meanwhile, nylon thread, predominantly used in the production of textiles, fabrics, and clothing items, generated a yield of 74.9 mol %. Nylon t-shirt fabric and 3D printing filament comprising 25 wt % glass fibers resulted in ε-caprolactam yields of 69.2 and 70.3 mol %, respectively.

To further assess the impurity tolerance of the developed methodology, both washed and unwashed fishing nets supplied by Bureo were evaluated. Washed fishing nets delivered a yield of 70.2 mol % to ε-caprolactam, whereas a slightly lower yield of 67.5 mol % achieved for the unwashed nets. This discrepancy suggests that the presence of impurities in the unwashed nets may slightly impair the deconstruction efficiency of the process. Finally, subjecting multilayer film containing 25 wt % of nylon 6 as well as a variety of other materials to our process resulted in a yield of 68.7 mol % to ε-caprolactam, further demonstrating the validity of our methodology toward mixed plastic waste.

### Process Modeling, TEA, and LCA of a Conceptual Nylon 6 Depolymerization Process

TEA and LCA were performed to evaluate the economics and environmental impacts of nylon 6 depolymerization via alcoholysis. Process modeling was accomplished using Aspen Plus v14.1, with the missing property data for the oligomeric compounds estimated using Aspen's property estimation tool; sensitivity and Monte Carlo simulations indicate that the overall conclusions of the TEA and LCA were not significantly affected by data availability. TEA used a discounted cash flow analysis approach to estimate the MSP. LCA was conducted with Brightway 2.9.7 software, employing the ReCiPe midpoint hierarchical method for impact assessment.

A simplified process flow diagram for the conceptualized process is shown in FIG. 14. In the base case scenario, the plant capacity was set to process 100 metric tons per day (MTPD), or 34,000 metric tons per year of nylon 6 waste, which represents 5% of 2022 nylon 6 consumption in North America. For process modeling, fishing nets were selected as the feedstock. While nets offer high feedstock quality compared to other nylon 6 waste streams, they also incur a higher cost for recovery. The feedstock is modeled as 96 wt % nylon 6 and 4 wt % impurities (modeled as sand and HDPE) sourced at $0.80/kg, which includes transportation costs ($0.65/kg) and collection and processing of the fishing nets ($0.15/kg). LCA data for the collection and pretreatment of post-use nylon 6 were not available in literature or in commercial databases. As a result, custom inventories were created by modifying known inventories for other post-consumer recycled plastics to include estimated transportation distance for post-use nylon fishing net and removal of irrelevant entries such as sorting at a materials recovery facility.

In the process, nylon 6 is shredded and washed to remove contaminants like sand and polyolefins. Following pretreatment, nylon 6 is extruded and introduced into the depolymerization reactor operating at 210° C. and 320 psi for 2.5 h. In the presence of H₃PO₄ as catalyst, and n-PrOH as solvent, nylon 6 undergoes depolymerization, yielding 77% ε-caprolactam and 23% oligomeric products. Following depolymerization, n-PrOH is recovered via distillation and recycled to the reactor, with over 99.5% n-PrOH recovery. The ε-caprolactam-rich bottom product of the n-PrOH recovery column is fed to a wiped-film evaporator. The oligomers-rich liquid stream from the wiped film evaporator is directed to an oligomer depolymerization kettle reactor, where 85% of the oligomers undergo depolymerization to ε-caprolactam. The ε-caprolactam is removed continuously from the top of the oligomer depolymerization reactor by stripping with superheated steam (steam to ε-caprolactam feed ratio of 5:1) and then recovered by distillation. After two distillation steps, the recovered ε-caprolactam stream is 97 wt % pure and is introduced into a VK column reactor. The recycled nylon 6 (r-nylon 6) product from the VK column is extruded into pellets and contacted with hot water in a counter-current extraction system to remove remaining ε-caprolactam and oligomers. The purified r-nylon 6 product is subjected to solid-liquid separation using a disc centrifuge and fed into a rotary dryer for moisture removal, resulting in a final composition of 99 wt % r-nylon 6 and 1 wt % water. Throughout the process, water and residual ε-caprolactam are recycled where possible. For example, unreacted ε-caprolactam from polymerization is recovered by distillation and recycled. When recovery is not possible, such as wash water from pretreatment and unreacted oligomers from the depolymerization reactor, the waste is neutralized with calcium hydroxide and sent to wastewater treatment.

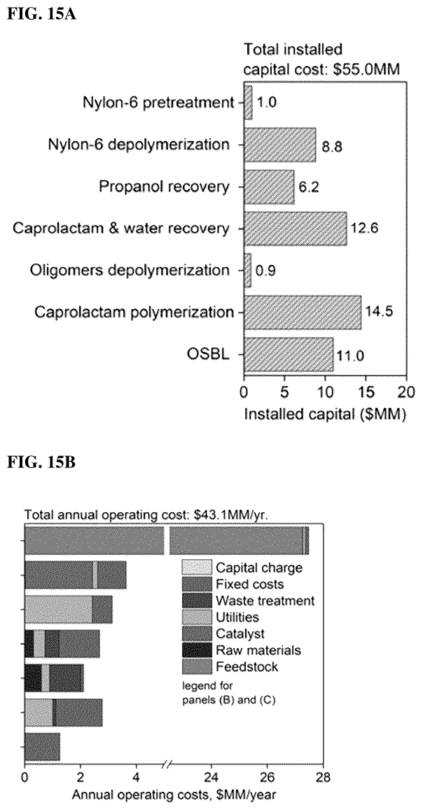

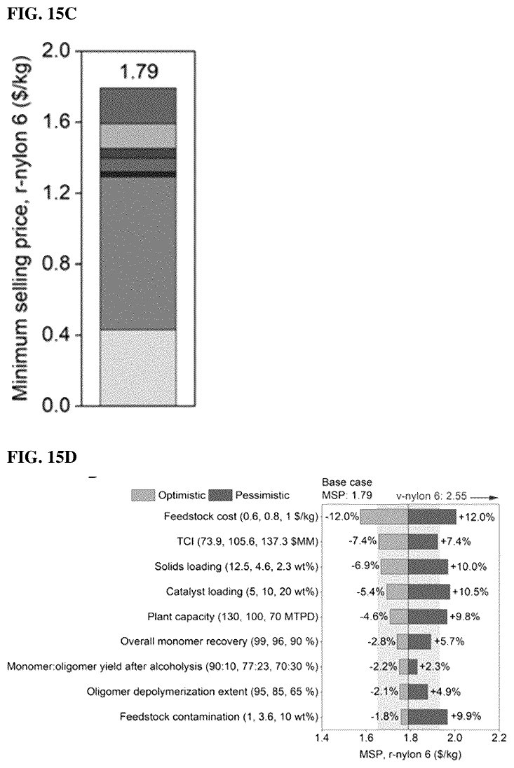

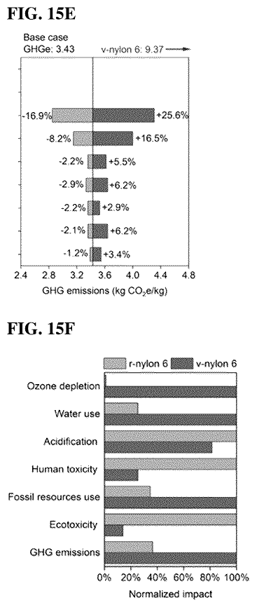

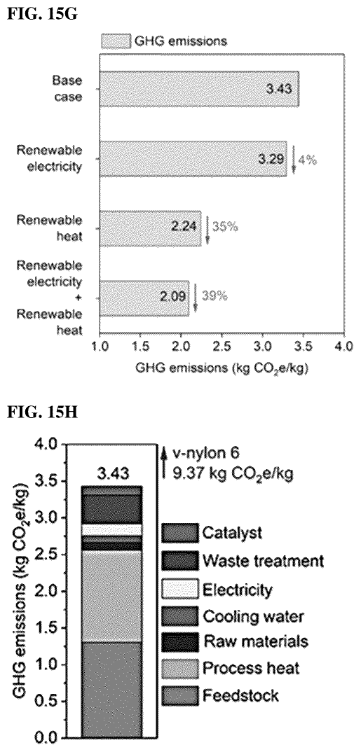

The CAPEX for the base case scenario was estimated as $105.6 million (MM) with a total installed cost of $55 MM [FIG. 15A]. The caprolactam polymerization and caprolactam & water recovery sections were the most CAPEX intensive, driven by the costs of the VK column reactor and distillation columns, accounting for $14.5 MM and $12.6 MM, respectively. The total annual OPEX was estimated to be $43.1 MM [FIG. 15B], including a feedstock cost of $27.3 MM (63%, purchased at $0.80/kg). The estimated CAPEX and OPEX for alcoholysis align with figures reported for comparable polymer recycling processes. Based on the CAPEX and OPEX estimates, a discounted cash flow rate of return analysis was performed to estimate the MSP of r-nylon 6 at $1.79/kg [FIG. 15C], with the two largest contributions coming from the feedstock (48% of overall MSP) and the capital charge (24% of overall MSP).

Recycled nylon 6 produced via alcoholysis is predicted to exhibit lower environmental impacts than virgin nylon 6 across multiple categories, including with 63%, 65%, and 75% reductions in GHG emissions, fossil resources use, and water use, respectively in large part due to the avoidance of primary caprolactam production, since nitrogen-containing chemicals such as caprolactam tend to be emissions-intensive. Conversely, life cycle impacts due to ecotoxicity, land use, and mineral resources, among several others are estimated to be higher, partly due to the electricity and process heat demand. Most of the environmental impacts of alcoholysis can be linked to the feedstock nylon 6 (9-89% contribution to impacts) mainly due to transportation, phosphoric acid (2-61%), process heat (6-41%), cooling water (0.5-25%), and electricity (1-48%). The other process consumables including n-PrOH, nitrogen, process water, and calcium hydroxide exhibit minimal environmental impacts. A second cradle-to-grave system boundary was considered to examine the effect of recycling nylon 6 multiple times via alcoholysis through a system expansion approach, which resulted in 13.7 potential lifetimes for an ideal recycling rate of 100%. Using lower recycling rates greatly reduces the achievable lifetimes (e.g., a recycling rate of 30% results in 1.4 lifetimes for the base case process). The calculated E-factor of 0.65 for the proposed plant is in the order of values expected for bulk chemicals and indicates substantial reduction in waste generation, thereby promoting resource efficiency and reduced ecological footprint.

To identify key process drivers, univariate sensitivity analysis was performed to investigate the impact on the MSP and environmental performance of alcoholysis as shown in FIGS. 15D-15E. Feedstock cost was the most sensitive parameter, with a 20% variation leading to a ±12% change in the MSP of r-nylon 6. However, a feedstock cost increase of up to $1.50/kg could be tolerated for r-nylon 6 to remain competitive with the historical market price of fossil-derived nylon 6 which has fluctuated between $1.63-$2.91/kg since 2010. Assuming no change in product yields, reducing solids loading to 2.3 wt % and increasing the catalyst loading to 20 wt % resulted in 6.9% and 5.4% increase in MSP, respectively, underscoring the economic burden of increased solvent and catalyst requirements. A lower plant capacity of 70 MTPD negatively impacted process economics, resulting in increased MSP by 9.8%. Changes in solids loading and catalyst loading had the biggest influence on the evaluated environmental impact categories, with the GHG emissions increasing by nearly 26% for low solids loading (2.3 wt %) [FIG. 15E] and human toxicity increasing by nearly 38% for higher catalyst loading (20 wt %). Feedstock impurities (up to 10 wt %) increased MSP and GHG emissions by 10% and 3.4%, respectively due to reduced product yields. Variations in reaction temperature did not affect MSP, as energy requirements for the alcoholysis reactor were met using surplus heat from other process units. Increased oligomer depolymerization extent (95%) marginally decreased MSP by 2.1% due to reduced oligomer losses in wastewater treatment. A decrease in the monomer-to-oligomer yield ratio in the alcoholysis reactor from 77%:23% (base case) to 70%:30% (pessimistic case) slightly reduced the overall ε-caprolactam yield from 95.6% to 94.7%, indicating a rather modest impact on process economics and environmental impacts. In addition, reducing the overall process monomer recovery to 90% led to a nearly 6% increase in MSP and GHG emissions. The solvent (n-PrOH) recovery rate was not considered as a sensitivity variable as even with partial solvent recovery, downstream processing necessitates complete solvent removal prior to ε-caprolactam polymerization to r-nylon 6. This additional downstream recovery step does not have a substantial impact on the overall economics compared to the baseline scenario with near-complete solvent recovery. Furthermore, a multivariate sensitivity analysis was carried out to understand how monomer yield after alcoholysis and solids loading affect the MSP of r-nylon 6. The results show that increasing both yield and solids loading leads to a lower MSP, with solids loading having a particularly strong impact. At low yield (50%) and low solids loading (3 wt %), MSP is approximately $1.86/kg whereas at yields above 90% and solids loadings above 14 wt %, MSP drops to about $1.70/kg. These insights further highlight the importance of optimising yield and solids loading to further improve process viability.

Based on this TEA and LCA, we propose several potential process improvements to further improve process feasibility [FIG. 14]. A key consideration is the purity of the feedstock. While a high-purity feedstock (high nylon 6 content and low contaminant content) can improve alcoholysis process efficiency, the associated cost premium might contradict with the low-cost feedstock requirement established in our MSP sensitivity analysis. Establishing a robust supply chain is thus essential. Implementing renewable energy sources for heating and electricity across the entire plant can further decrease net GHG emissions by 39% [FIG. 15G]. To enhance the overall process efficiency, the depolymerization step benefits from maximum yields. While the influence of changes in the monomer-to-oligomer yield ratio on the MSP is relatively minor, achieving monomer yields of 80% or higher is desirable to reduce the need for energy-intensive oligomer separation. By increasing solids loading to 12 wt % and beyond, solvent requirements can be minimized, thereby reducing costs and associated environmental burdens.

When considering the scale-up potential of new technologies, it is important to avoid burden shifting. Therefore, in addition to examining the costs and environmental impacts of nylon 6 depolymerization, the environmental risk implications of this technology were investigated using the framework developed by Uekert et al. The analysis revealed that the alcoholysis process does not involve toxic chemicals, or chemicals that have notable human health or environmental risks. However, the upstream processes associated with the collection, cleaning, and sorting of plastic waste utilizes sulfuric acid, which is identified as a toxic chemical, and sodium hydroxide, which is highly corrosive. Although ferric chloride and sodium hydroxide are used in cleaning steps and can be toxic to aquatic organisms, their concentrations remain well below toxic levels.

### CONCLUSIONS

Acid-catalyzed alcoholysis is a mild, effective strategy for the depolymerization of nylon 6, resulting in the selective production of ε-caprolactam at excellent yields. Subsequent analyses reveal that the depolymerization of nylon 6 with n-PrOH and H₃PO₄ proceeds through a consecutive reaction sequence consisting of an alcoholysis-driven depolymerization step followed by rapid cyclodealcoholization of the formed propyl 6-aminohexanoate intermediate. Interestingly, the latter cyclodealcoholization step appears to exhibit noticeable equilibrium limitations when utilizing n-PrOH as a solvent. This catalytic protocol is effective for the depolymerization of a variety of real-world nylon 6 substrates including fishing net, 3D printing filament, film, fabric, carpet, thread, and contaminated used fishing net, allowing for their selective conversion to ε-caprolactam at yields as high as 76%. Subsequent TEA estimates a minimum selling price (MSP) of recycled nylon 6 polymer generated via our process of $1.79/kg, 30% lower than the 5-year average market price of virgin nylon 6. LCA further indicates that this material could result in GHG emissions reductions of over 60% compared to primary production, with sensitivity analyses revealing that solids and catalyst loading are the key cost & GHG drivers for the process. Overall, our process addresses a variety of challenges associated with the recycling of nylon 6 and provides a sustainable and resource-efficient route for its chemical recycling, thereby providing a viable path to a circular nylon economy.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

1. A method for producing ε-caprolactam from nylon 6, comprising contacting the nylon 6 with an alcohol and an acid.

2. The method of claim 1, wherein the alcohol is methanol, ethanol, n-propanol, isopropanol, n-butanol, or ethylene glycol.

3. The method of claim 1, wherein the alcohol is propanol.

4. A method for producing ε-caprolactam from nylon 6, comprising contacting the nylon 6 with ammonia and an acid.

5. The method of claim 4, wherein the ammonia is at a pressure of more than 60 psi.

6. The method of claim 4, wherein the ammonia is at a pressure of about 60 psi, about 80 psi, about 100 psi, or about 120 psi.

7. The method of claim 4, wherein the ammonia is at a pressure of about 80 psi.

8. The method of claim 1, wherein the nylon 6 is nylon 6 plastic waste.

9. The method of claim 8, wherein the plastic waste is nylon 6 powder, nylon 6 film, nylon 6 pellets, green nylon 6 fishing net, white nylon 6 fishing net, nylon 6 thread, nylon 6 clothing, nylon 6 carpet, or nylon 6 filaments.

10. The method of claim 1, wherein the acid is a Lewis acid.

11. The method of claim 1, wherein the acid is a Brønsted acid.

12. The method of claim 1, wherein the acid is a mineral acid.

13. The method of claim 1, wherein the acid is HCl, H₂SO₄, H₃PO₄, (NH₄)₂HPO₄, Sn(OTf)₂, Ce(OTf)₄, or La(OTf)₃.

14. The method of claim 13, wherein the acid is phosphoric acid (H₃PO₄).

15. The method of claim 1, wherein the acid is present about from 1 wt % to about 25 wt %.

16. (canceled)

17. The method of claim 1, wherein the method is performed in a protic solvent.

18. The method of claim 1, wherein the method is performed in an alcoholic solvent.

19. The method of claim 1, wherein the method is performed in methanol, ethanol, n-propanol, isopropanol, n-butanol, or ethylene glycol.

20. The method of claim 19, wherein the method is performed in n-propanol.

21-25. (canceled)

26. The method of claim 1, wherein the method produces ε-caprolactam in a yield of about 70%, about 75%, about 80%, about 85%, or about 90% compared to the amount of nylon 6 used.
